(19) United States
(12) Patent Application Publication
(10) Pub. No.: US 2007/0190024 A1
(43) Pub. Date: Aug. 16, 2007

(54) Il-11 muteins

(75) Inventors: Francois Emmanuel Paris, Nantes (FR); Yannick Jacques, Nantes (FR); Jean Content, Saint Genese (BE); Xiao-Ming Wang, Bruxelles (BE); Dimitri Harmegnies, Bruxelles (FR); Joachim Grotzinger, Altwitlenbek (DE)

Correspondence Address: NIXON & VANDERHYE, PC, 901 NORTH GLEBE ROAD, 11TH FLOOR, ARLINGTON, VA, 22203, US

(73) Assignee: INSERM Institut National de la Sante et de la Rech, Paris Cedex 13 (FR)

(21) Appl. No.: 10/565,626

(22) PCT Filed: Jul. 29, 2004
(86) PCT No.: PCT/EP04/09165

§ 371 Date: Nov. 20, 2006

Related U.S. Application Data
(60) Provisional application No. 60/490,525, filed on Jul. 29, 2003.

(51) Int. Cl. A61K 38/20; A61K 48/00; C07K 14/54; C07H 21/04; C12P 21/04

(52) U.S. Cl. 424/85.2; 530/351; 435/69.52; 536/23.5; 435/320.1; 435/325; 514/44

(57) ABSTRACT

The present invention relates to new IL-11 muteins of which site I hydrophobocity has been increased. These muteins act as IL-11 agonist or hyperagonist, and are notably useful as anti-thrombocytopenia agents, and as agents improving the resistance of an organism to the deleterious in vivo effects induced by radiation or chemotherapy during the treatment of cancer or for the preparation of patient to transplantation.

### BACKGROUND OF THE INVENTION

Human interleukin-11 (hIL-11) is a multi-potential cytokine that is involved in numerous biological activities such as hematopoiesis, osteoclastogenesis, neurogenesis and female fertility. It also displays anti-inflammatory properties. hIL-11 is clinically used to treat chemotherapy-induced thrombocytopenia.

Interleukin-11 was cloned from the primate stromal cell line PU-34 and was initially considered as a haematopoietic cytokine. It was later found that it also has effects on non-haematopoietic systems and that it acts on many different cell types and tissues. Numerous experiments on animal models and clinical trials with patients suffering from acute and chronic inflammatory diseases, including rheumatoid arthritis, inflammatory bowel disease, inflammatory liver disease, mucositis and psoriasis have revealed that IL-11 is an anti-inflammatory and mucosal protective agent, which, by inhibiting nuclear translocation of nuclear factor-κB (NF-κB), can reduce the production of pro-inflammatory cytokines secreted by macrophages such as TNF-α, IL-1β, IL-6 and IL-12. Its radio-protective and septic shock-protective activities have also been demonstrated in other experiments. The clinical application of hIL-11 has been approved by the FDA for the treatment of chemotherapy-induced thrombocytopenia due to the ability of this cytokine to stimulate megakaryocytopoiesis and thrombopoiesis. Another potential therapeutic application of IL-11 in the treatment of mild hemophilia A or von Willebrand disease was recently evidenced by the fact that IL-11 is able to increase von Willebrand factor and factor VIII production in a von Willebrand disease mouse model as well as in healthy mice.

Because of its broad spectrum of action, improved agonists as well as IL-11 antagonist would be of interest for numerous biological and clinical applications.

Some structure studies of IL-11 molecule have been conducted to elucidate the interactions involved in IL-11 activation and signalling.

The structure study of Czupryn et al. 1995 thus describes the production of 61 mutated forms of hIL-11 from E. coli as thioredoxin fusion proteins [Czupryn et al. (1995) Alanine-scanning mutagenesis of human interleukin-11: identification of regions important for biological activities. Ann. New York Acad. Sci. July 21; 762, 152-164]. Testing of these mutated forms in a murine T10 plasmacytoma proliferation assay led to the conclusion that mutations made several positions proximal to the hIL-11 C-terminus, such as a D186A mutation, caused substantial reduction in biological activity (the D186A mutation induced a 500-fold decrease in biological activity on the murine plasmacytoma cell line T10), and that a number of other mutations in this region affected either protein folding or stability.

Tacken et al. 1999 have built a three-dimensional model of human IL-11 [Tacken et al. (1999) Definition of receptor binding sites on human interleukin-11 by molecular modelling-guided mutagenesis. Eur. J. Biochem. 265, 645-655]. Three receptor binding sites within the IL-11 molecule have thus been defined (see site I, site II and site III on FIG. 1B of Tacken et al. 1999).

In Tacken et al. 1999 study, ten surface-exposed amino acid have thus selected within sites I, II and III as candidate for single point mutagenesis assays (only one amino acid per molecule has been mutated). The single point mutations made consisted in replacing a hydrophobic side chain by a charged group (aspartic acid), and a charged chain by an oppositely charged residue (lysine or glutamic acid) in order to introduce a substantial disturbance into the receptor binding sites. Nine of the ten single point mutants thus produced, including those four for which the single point mutation was on an amino acid belonging to site I (A84D mutant; L85D mutant; R190E mutant; L194D mutant), led to a substantially reduced affinity for the IL-11 receptor complex, and to a loss or a substantially reduced bioactivity (loss or substantial decrease in induction of α1-anti-chymotrypsin synthesis in HepG2 cells, and of proliferation of Ba/F3-130-11α cells). Only one of the mutants, namely R135E, which results from the replacement of a site II hydrophilic amino acid by a still hydrophilic but oppositely-charged amino acid, appeared to potentially constitute a hyperagonistic IL-11 mutant.

There thus remains a need for a method to efficiently produce IL-11 agonists, and to obtain IL-11 agonists that would prove to be active in vivo.

### SUMMARY OF THE INVENTION:

The inventors have designed and produced IL-11 muteins wherein the hydrophobicity at site I has been substantially increased by replacement of at least two IL-11 site I hydrophilic amino acids by hydrophobic counterparts. The muteins have been characterized in terms of structure, affinity, specificity and bioactivity. Electrophoretic analysis, gel filtration, infrared spectroscopy and circular dichroism indicate that these new proteins are more compact than wild-type IL-11.

The IL-11 muteins of the invention bind to IL-11Rα with an enhanced affinity (a three-fold enhanced affinity has been measured) and retain the ability to recruit gp130 through site II.

As an advantageous feature, they retain the ability to induce in vitro proliferation of various IL-11 dependent cells. A mutein of the invention; namely the H182V+D186A hIL-11 mutein, has further been shown to be 60 to 400 fold more active than wild-type IL-11 on the in vitro proliferation of 7TD1 murine hybridoma cells.

The muteins of the invention also advantageously retain in vivo biological activity. Their in vivo biological activity can further be much higher than wild-type IL-11. An injection of the H182V+D186A hIL-11 mutein at a 10-fold lower dose than the wild-type hIL-11 has been shown to delay the death of irradiated mice for the same duration.

Compared to single-point mutation IL-11 muteins such as a D186A IL-11 mutein, the double point muteins of the invention prove to have in vitro and in vivo unexpected effect and advantages. They notably induce much higher survival rates upon exposure to radiation (i.e. upon inhibition of microvascular endothelial apoptosis); see comparative illustrative data described in example 3 below, and enclosed FIGS. 35-36.

The muteins of the invention are therefore useful in every biological, medical or clinical application in which wild-type IL-11 is useful, and can even show an enhanced efficiency. The muteins of the invention are more particularly useful in radioprotection (e.g. radioprotection of the small intestine during abdominal irradiation), in decreasing chemotherapy deleterious effects (e.g. during 5-fluoroUracil chemotherapy), in anti-inflammatory therapy, in resistance to septic shock, to diabetes, and in hematopoiesis stimulation.

### BRIEF DESCRIPTION OF THE DRAWINGS

FIG. 1 is a reprint of AY207429 accession entry from the NCBI website (htt://www.ncbi.nlm.nih.gov/entrez) giving the nucleotide and amino acid wild-type human IL-11 (hIL-11) sequences and characteristics (SEQ ID NO:73, and SEQ ID NO:1, respectively).

FIG. 2 shows the complete wild-type human, macaque, mouse and rat IL-11 amino acid sequences (SEQ ID NO:1-4).

FIG. 3 shows the wild-type human, macaque, mouse and rat IL-11 amino acid sequences deleted from the first 34 N-terminal amino acids (SEQ ID NO:5-8). H182 and D186 are underlined.

FIG. 4 shows hIL-11 muteins of the invention (SEQ ID NO:9-13), which derive from the 34aa-deleted wild-type hIL-11 by replacement of the wild-type H182 and D186 by hydrophobic amino acids (shown underlined).

FIG. 5 shows hIL-11 muteins of the invention (SEQ ID NO:14-18), which derive from wild-type hIL-11 deleted from its first 21 amino acids, by replacement of the wild-type H182 and D186 by hydrophobic amino acids (shown underlined).

FIG. 6 shows hIL-11 muteins of the invention (SEQ ID NO:19-23), which derive from complete wild-type hIL-11, by replacement of the wild-type H182 and D186 by hydrophobic amino acids (shown underlined).

FIG. 7 shows IL-11 muteins of the invention (SEQ ID NO:24-28), which derive from 34aa-deleted wild-type macaque IL-11, by replacement of the wild-type H182 and D186 by hydrophobic amino acids.

FIG. 8 shows IL-11 muteins of the invention (SEQ ID NO:29-33), which derive from the wild-type macaque IL-11 deleted from the first 21 N-terminal amino acids, by replacement of the wild-type H182 and D186 by hydrophobic amino acids.

FIG. 9 shows IL-11 muteins of the invention (SEQ ID NO:34-38), which derive from complete wild-type macaque IL-11, by replacement of the wild-type H182 and D186 by hydrophobic amino acids.

FIG. 10 shows IL-11 muteins of the invention (SEQ ID NO:39-43), which derive from the wild-type mouse IL-11 deleted from the first 34 N-terminal amino acids, by replacement of H182 and D186 by hydrophobic amino acids (shown underlined).

FIG. 11 shows IL-11 muteins of the invention (SEQ ID NO:44-48), which derive from the wild-type mouse IL-11 deleted from the first 21 N-terminal amino acids, by replacement of H182 and D186 by hydrophobic amino acids (shown underlined).

FIG. 12 shows IL-11 muteins of the invention (SEQ ID NO:49-53), which derive from the complete wild-type mouse IL-11, by replacement of H182 and D186 by hydrophobic amino acids (shown underlined).

FIG. 13 shows IL-11 muteins of the invention (SEQ ID NO:54-58), which derive from the wild-type rat IL-11 deleted from the first 34 N-terminal amino acids, by replacement of H182 and D186 by hydrophobic amino acids (shown underlined).

FIG. 14 shows IL-11 muteins of the invention (SEQ ID NO:59-63), which derive from the wild-type rat IL-11 deleted from the first 21 N-terminal amino acids, by replacement of H182 and D186 by hydrophobic amino acids (shown underlined).

FIG. 15 shows IL-11 muteins of the invention (SEQ ID NO:64-68), which derive from the complete wild-type rat IL-11, by replacement of H182 and D186 by hydrophobic amino acids (shown underlined).

FIG. 16A shows the joined CDS sequence (SEQ ID NO:69) for human complete wild-type IL-11, as defined in AY207429 NCBI nucleotide entry, and the joined CDS sequence (SEQ ID NO:70) for the hIL-11 muteins of the invention which derive from the 34aa-deleted hIL-11.

FIG. 16B shows the joined CDS sequence (SEQ ID NO:71) for the hIL-11 muteins of the invention which derive from the 21aa-deleted hIL-11, and the joined CDS sequence (SEQ ID NO:72) for the hIL-11 muteins of the invention which derive from the complete hIL-11.

FIG. 17 shows the AY207429 NCBI entry nucleotide sequence mutated in accordance with the present invention (codons n₁n₂n₃ and n₄n₅n₆ which replace the wild-type cac and gac are shown underlined).

FIG. 18 shows the mRNA sequence (SEQ ID NO:75) of a mutein of the invention, which derives from hIL-11(codons n₁n₂n₃ and n₄n₅n₆ are shown underlined).

FIG. 19 shows the gene sequence (SEQ ID NO:76) of a IL-11 mutein of the invention which derive from hIL-11(codons n₁n₂n₃ and n₄n₅n₆ are shown underlined).

FIG. 20 shows the % of mouse survival par days of exposure to irradiation at 15 Gy (upper curve=mice treated with 3.2 micrograms of recombinant but non-mutated IL-11; lower curve=non-treated control mice).

FIG. 21 shows the % of mouse survival per days of exposure to irradiation at 15 Gy (upper curves=mice treated with 3.2 micrograms of recombinant but non-mutated IL-11, or with 0.32 microgram of a H182V+D186A mutein of the invention—“HVDA”−; lower curves=mice treated with 0.32 microgram of recombinant but non-mutated IL-11, or non-treated control mice).

FIG. 22 shows the parental (non-mutated) nucleotide sequence (SEQ ID NO:77) of a recombinant IL-11 (FPΔIL-11), and its parental (non-mutated) amino acid sequence (SEQ ID NO:78).

FIG. 23 shows the nucleotide sequence of FPΔIL-11 mutated in accordance with the present invention (SEQ ID NO:79), and the mutated corresponding amino acid sequence (SEQ ID NO:80 of the invention)

FIG. 24 shows the primers used for inverse PCR mutagenesis of FPΔIL-11.

FIGS. 25A and 25B show a human wild-type Il-11 3D-model.

In FIG. 25A, a 3D model of the IL-11 based on cristallographic data obtained for CNTF, as described by Tacken et at [1999] is shown. FIG. 25B shows a site I view of the IL-11 model. Positively charged amino acids (Arg, Lys) are coloured in blue, negatively charged (Asp, Glu) are in red, hydrophilics in grey and hydrophobic in yellow.

FIG. 26 shows the expression of FPΔIL-11 and of the H182V+D186A mutein analysed by SDS-PAGE.

BL21 E. coli were transformed with pET-22b(+) vector encoding FPΔIL-11 and H/V-D/A mutein or empty vector (E). After induction (i, induced; n, not induced) of protein production, bacteria were lysed as described in Experimental. Supernatants (100 μg of total protein per lane) were then analysed by SDS-PAGE and colored by Coomassie blue.

FIG. 27 shows infrared spectra of FPΔIL-11 (top) and of H182V+D186A (bottom) in the 1800-1400 cm⁻¹ frequency range.

The absorbance is reported in mOD. The absorbance scale is given for the bottom spectrum. The upper spectrum has been offset for clarity.

FIG. 28 shows the CD spectra of the FPΔIL-11 (top) and of H182V+D186A (bottom).

FIGS. 29A and 29B show the evolution of the integrated intensity of the amide II band as a function of the time of exposure to ²H₂O for FPΔIL-11 (circles) and for H182V+D186A (crosses).

Fitting was carried out with a three exponential decay. Panel 29A: between 0 and 20 min, panel 29B: between 0 and 700 min.

FIGS. 30A and 30B show gel-filtration chromatography of parental FPΔIL-11 and of mutant H182V+D186A, and their bioactivity tested from fractions collected during the chromatography.

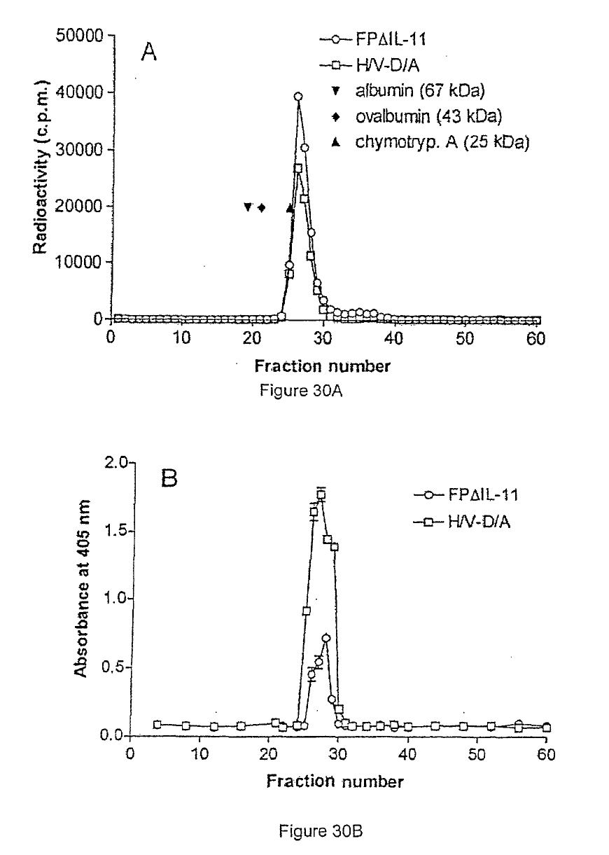

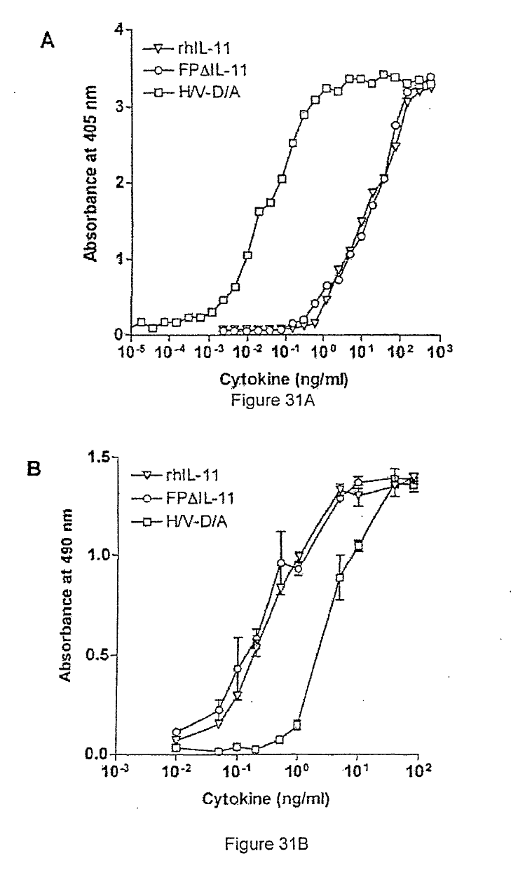

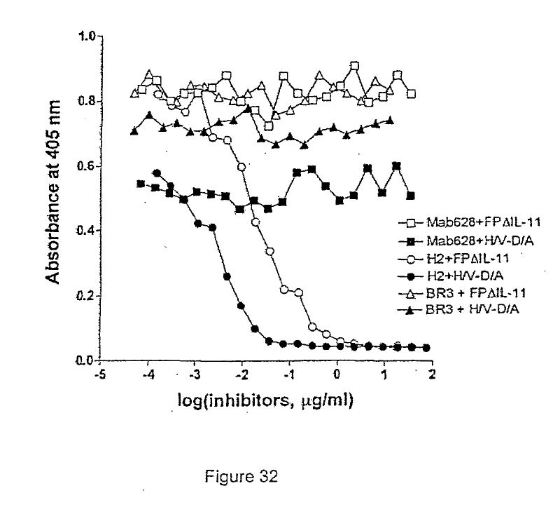

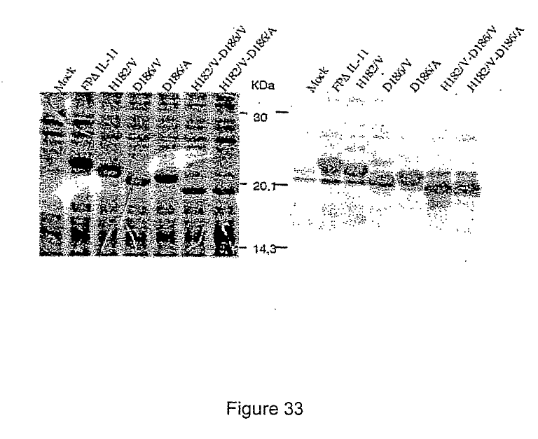

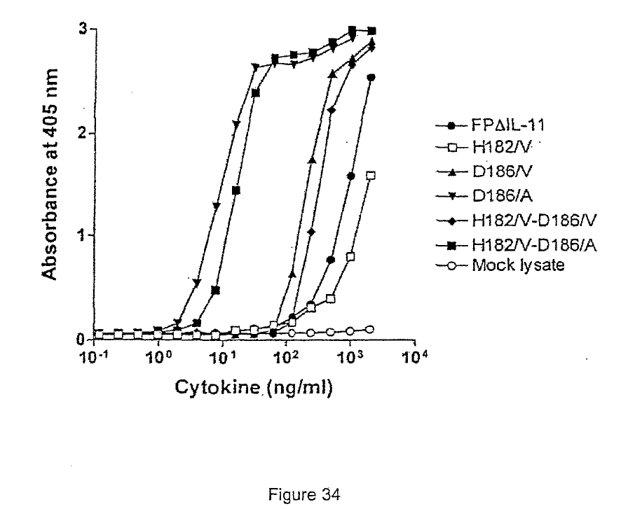

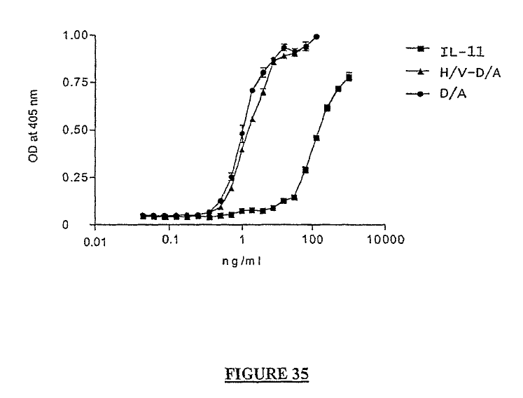

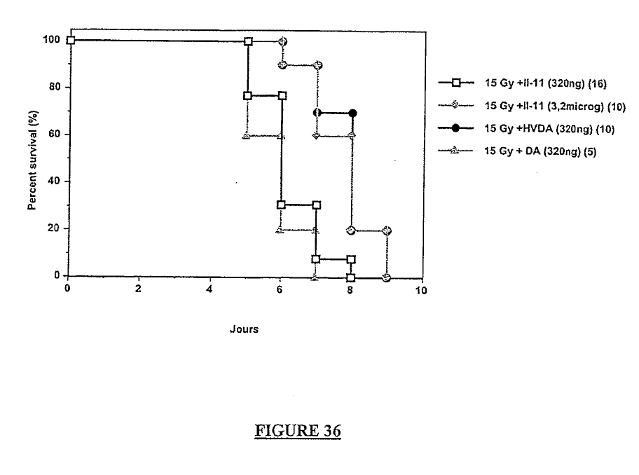

In FIG. 30A, Superdex-75 column (K16, Pharmacia Biotech) was used and calibrated with three proteins albumin (67 kDa), ovalbumin (43 kDa) and chymotrypsinogen A (25 kDa) before loading 30 μg of each analysed unlabelled protein in the presence of 50 ng of the same ³³P-labelled one as a tracer. Fifty microlitres of each collected fraction was submitted to a radio-counting. In FIG. 30B, IL-11 activity was measured using the mouse hybridoma cell line 7TD1. Cells were cultivated in flat-bottom microwell plates (2×10³ of 7TD1 cells/well) in the presence of 0.2 μl of each eluted fraction. After 7 days of culture, the number of surviving cells was determined by colorimetric assays for hexosaminidase. Each sample was tested in triplicate and presented on average with a standard deviation.

FIGS. 31A and 31B show the bioactivity of parental FPΔIL-11 and mutant H182V+D186A, tested on 7TD1 (FIG. 31A) and on B9 cells (FIG. 31B).

Cells were cultivated in flat-bottom microwell plates (2×10³ of 7TD1 cells/well; 1×10⁴ of B9 cells/well) in the presence of serial dilutions of parental FPΔIL-11, mutein H/V-D/A, or commercial rhIL-11 (R&D). After 7 days for 7TD1 and 3 days for B9 cells culture, the number of surviving cells was determined by colorimetric assays for hexosaminidase (7TD1 cells) and for XTT (B9 cells). Each sample was tested in triplicate and presented as average with a standard deviation.

FIG. 32 shows the inhibition of 7TD1 cells proliferation stimulated by FPΔIL-11 or mutant H182V+D186A, by anti-hIL-11 and anti-human gp130 neutralizing antibodies. Cells were incubated with the indicated concentrations of anti-human IL-11 monoclonal antibodies H2 (circles) and anti-human gp130 monoclonal antibodies MAB628 (squares) and B-R3 (triangles). Data points represent the means of triplicate determinations.

FIG. 33 shows the expression of parental FPΔIL-11 and of its H182V+D186A mutein analysed by SDS-PAGE and immunoblotting.

BL21 E. coli were transformed with empty vector (mock) or expression vector encoding parental FPΔIL-11 or mutated proteins as indicated in the figure. After induced expression of the proteins, cells were lysed by sonication and lysates (100 μg of total protein per lane) were analysed by SDS-PAGE (left) and immunoblotting (right) using a polyclonal antibody raised against IL-11 (B1AF 218).

FIG. 34 shows the proliferation of 7TD1 cells in response to FPΔIL-11 and its H182V+D186A mutein.

7TD1 cells were incubated in the presence of serial dilutions of E.coli lysate containing mock, FPΔIL-11 or muteins, which were previously adjusted to 2 μg/ml. After 7 days of culture, the number of cells was determined by a colorimetric assay for hexosaminidase.

FIG. 35 gives an in vitro comparative demonstration of an advantageous aspect of the muteins of the invention, by showing the respective proliferation levels shown by 7TD1 cells (ICLC accession number=HYL96001) in response to increasing doses of wild-type IL-11 (square symbols), or of H/V-D/A muteins of the invention (triangle symbols), or of D/A muteins (circle symbols).

FIG. 36 gives an in vivo comparative demonstration of an advantageous aspect of the muteins of the invention, by showing the respective percent survivals of 1.5 Gy/min irradiated mice after injection of IL-11 at 320 ng (square symbols) or at 3.2 microgrammes (lozenge symbols), or after injection of the H/V-D/A muteins of the invention at 320 nanogrammes (circle symbols), or after injection of D/A muteins at 320 nanogrammes (triangle symbols).

### DETAILED DESCRIPTION

IL-11 signalling is at present time known to be dependent on the formation of a ligand/receptor complex which comprises IL-11, IL-11Rα0 subunit (responsible for the specificity of interaction) and gp130 receptor β-subunit (responsible for signal transduction). The interaction between IL-11 and its receptor α-subunit occurs at its recently assigned site I (Tacken et al. 1999, cited supra, and incorporated herein by reference).

Activity of IL-11 requires binding to α receptor subunit (IL-11Rα) that provides ligand specificity in a functional multimeric signal-transduction complex with gp130, the common receptor subunit for the cytokine family including IL-6, vIL-6, CNTF (Ciliary Neurotrophic Factor), LIF (Leukaemia Inhibitory factor), OSM (Oncostatin M), CT-1 (Cardiotrophin) and NNT-1/BSF-3 (Novel neurotrophin-1/B cell-stimulating factor-3). It is believed that IL-11 first interacts with IL-11Rα with a low affinity (K_{d}=10 nM) and that the IL-11/IL-11Rα complex subsequently interacts with gp130 to form a high affinity (K_{d}=300-800 pM) and signal-transducing complex.

Three sites, responsible for the interaction with the receptor subunits have been assigned for IL-11 [Grotzinger, J., Kurapkat, G., Wollmer, A., Kalai, M. and Rose-John, S. (1997) The family of the IL-6-type cytokines: specificity and promiscuity of the receptor complexes. Proteins 27, 96-109; Tacken, I., Dahmen, H., Boisteau, O., Minvielle, S., Jacques, Y., Grotzinger, J., Kuster, A., Horsten, U., Blanc, C., Montero-Julian, F. A., Heinrich, P. C. and Muller-Newen, G. (1999) Definition of receptor binding sites on human interleukin-11 by molecular modelling-guided mutagenesis. Eur. J. Biochem. 265, 645-655].

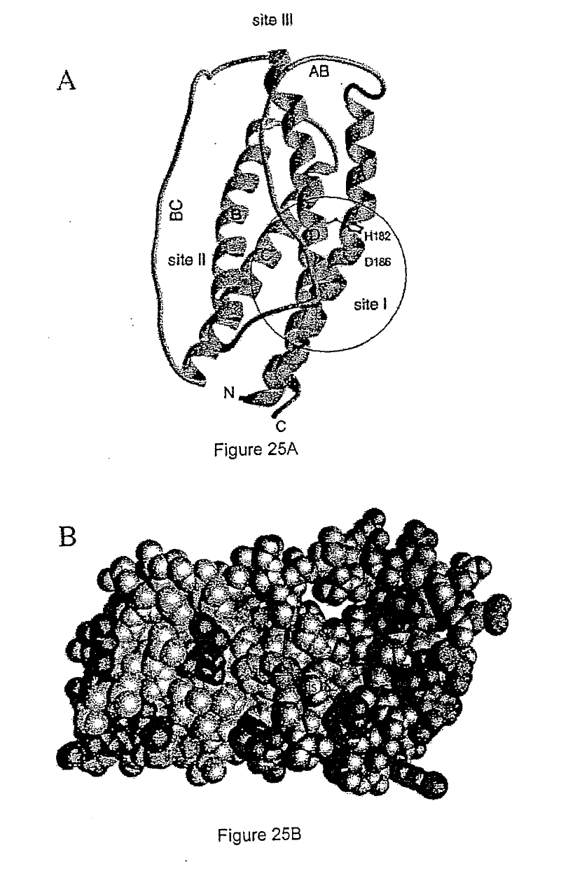

Site I, which essentially consists of amino acids at the end of the AB-loop and the C-terminal part of the D-helix, is implicated in the interaction with the IL11Rα subunit. Site II, which essentially consists of amino acids from the A and C helices and site III, which essentially consists of the N-terminal part of the D-helix and residues from the beginning of the AB-loop, are responsible for gp130 (β-subunit) recruitment (FIG. 25).

The inventors found that IL-11 muteins can be produced that have an increased affinity for IL-Rα, that have retained affinity for gp130, and that have retained or improved IL-11 biological activity.

The inventors demonstrate that such agonistic IL-11 muteins are obtainable by substantially increasing the hydrophobicity of IL-11 site I, which thereby makes the structure of the IL-11 molecule more compact. Increasing the hydrophobicity of IL-11 site I can be achieved by replacement of IL-11 site I hydrophilic amino acids by hydrophobic counterparts. It further appeared to the inventors that at least two of such hydrophilic amino acids should be replaced by hydrophobic counterparts.

The inventors notably demonstrate that site I of human wild-type IL-11 comprises two hydrophilic amino acids (His182 and Asp186), and that substituting both of them by hydrophobic counterparts (e.g. substituting His182 and Asp186 by Valine and Alanine, respectively) leads to a hIL-1 mutein with increased affinity for IL-11Rα, increased specificity and increased in vitro and in vivo bioactivity.

The fact that the muteins of the invention have such excellent properties and effects is all the more surprising and unexpected since opposite properties and effects are obtained when only one of these two hydrophilic amino acids is substituted. Indeed, Czipryn et al. 1995 (cited supra) describes that substituting D186 by A (without substituting H186) results in a human IL-11 mutein which has, with respect to the wild-type human Il-11, a highly decreased biological activity: a 500 fold decrease in biological activity has been measured on murine plasmacytoma cell line T10. This document hence disclosed the D/A mutation as a highly undesired candidate mutation to obtain a mutein with increased biological activity.

In fact, the inventors now further demonstrate that one cannot directly rely on results obtained in vitro from cell lines to reliably assume that a given mutation is a good or a bad candidate to obtain a mutein with increased efficiency: the same D186A mutation, but on a FPΔIL-11 protein (Flag tag+deletion of N-terminal proline-rich region, see the examples below), has been assayed by the inventor on another cell line (cell line 7TD1, see the examples below), and appears to induce an increase in biological activity for this cell line.

As a matter of fact, the inventors have performed in vivo experiments which confirm that, compared to single-point mutation IL-11 muteins such as a D186A IL-11 mutein, the double point muteins of the invention prove to have in vivo unexpected effect and advantages. They notably induce much higher survival rates upon exposure to radiation (i.e. upon inhibition of microvascular endothelial apoptosis); see comparative illustrative data described in example 3 below, and enclosed FIGS. 35-36.

It can also be noted that a mutein of the invention for which both His182 and Asp186 are mutated (by Val and Ala, respectively) is surprisingly and unexpectedly a lot more biologically active than human wild-type IL-11: a 60 to 400 fold increase in in vitro cell proliferation has been measured on 7TD1 murine hybridoma cells. Furthermore, as a very advantageous and in fact highly essential feature, the muteins of the invention induce an increase of biological activity in vivo in an animal, such as a mammal (a 10-fold increase in radioprotection in vivo efficiency has been measured on irradiated mice with H/V-D/A, see the examples below).

The present invention thus provides with a method to produce an IL-11 agonist, which comprises producing an IL-11 mutein wherein site I hydrophobicity has been increased by replacement of at least two non-hydrophobic amino acids which are part of the wild-type IL-11 epitope for IL-11Rα by hydrophobic ones.

As said two non-hydrophobic amino acids are part of the wild-type IL-11 epitope for IL-11Rα, they belong to what is known as IL-11 site I (=end of AB-loop and C-terminal part of the D-helix).

Said at least two non-hydrophobic amino acids most preferably are surface-exposed.

The mutein molecule is thereby rendered more compact. It has retained the ability to bind to IL-Rα through its mutated site I, and has also retained the ability to bind to the other components of the IL-11 signal transducing complex, and notably to gp130 through site II and site III of the mutein.

It has also retained the ability to induce in vitro proliferation of IL-11 dependent cells, such as 7TD1 murine hydridoma cells available from the ICLC (Interlab Cell Line Collection of the Istituto Nazionale per la Ricerca sul Cancro; L. go R. Benzi, 10; 16132 Genova, Italy; see http://www.iclc.it/Lista.html and http://www.biotech.ist.unige.it; ICLC Catalogue accession number=HYL96001).

It has also retained in vivo bioactivity, such as e.g. the ability to protect against radiation.

As the muteins of the invention have at least retained IL-11 affinity and bioactivity, they can be referred to as IL-11 agonist or hyperagonist.

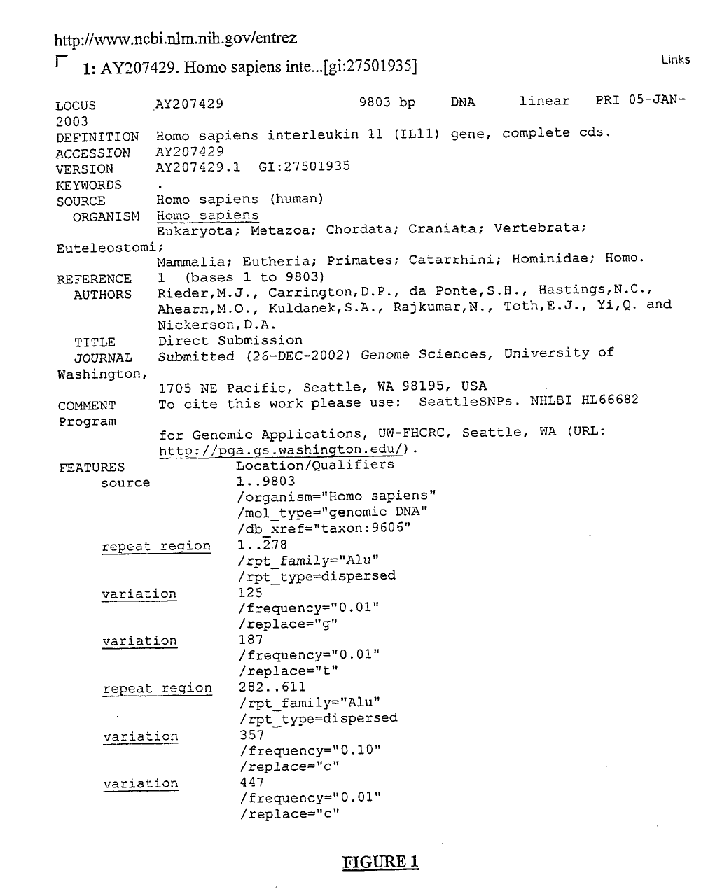

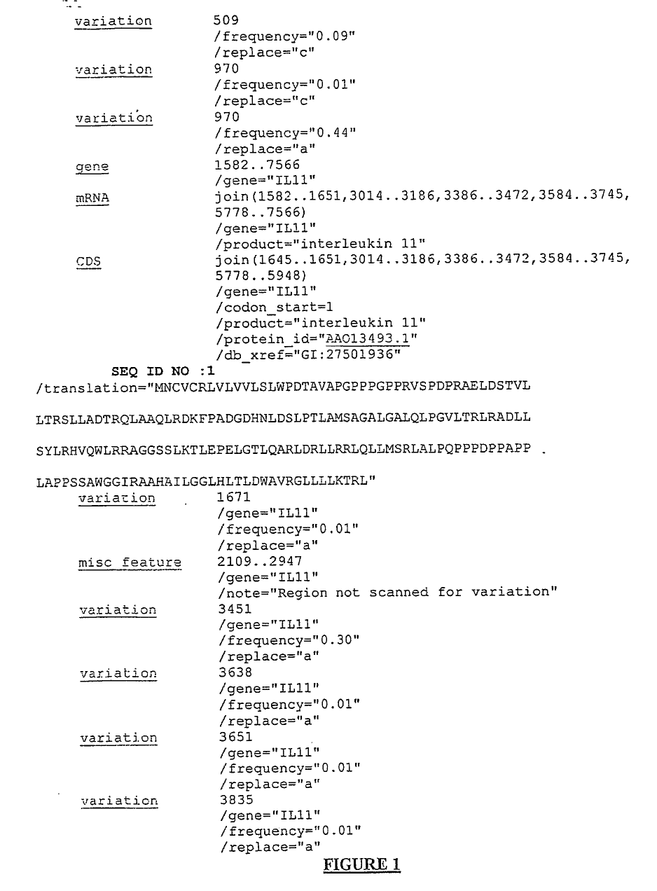

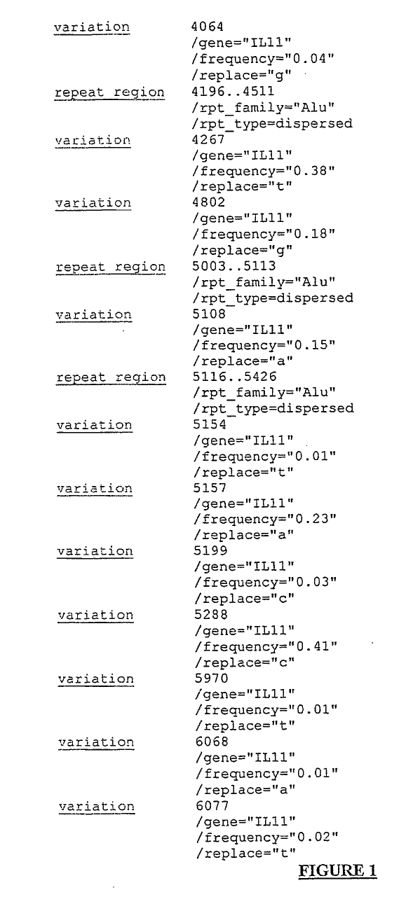

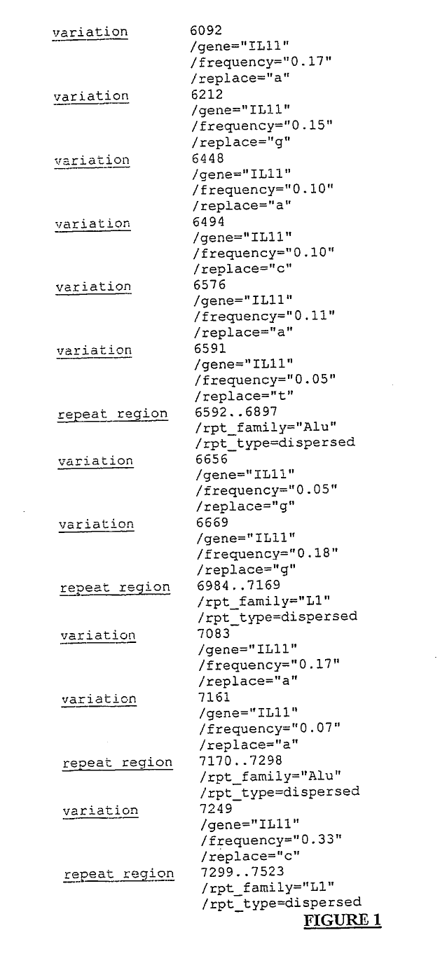

To determine whether a given IL-11 amino acid is or not part of the epitope for IL-11Rα, and whether it is or not surface-exposed, the person of ordinary skill in the art can proceed in line with what is described in Tacken et al. 1999 (cited supra). It may e.g. comprise the use of a three-dimensional structure representation of said IL-11 to locate said given amino acid so that it can be determined whether it belongs or not to site I (=epitope for IL-11Rα) and whether it is or not surface-exposed (see FIGS. 1A and 1B of said Tacken et al. 1999, as well as the section within this publication which is entitled “Generation of a molecular model of interleukin-11 and selection of a amino acid residues for site-directed mutagenesis”, the content of which is herein incorporated by reference).

Nucleotide and amino acid sequences of wild-type IL-11Rα are available from standard sequence databanks known to the person of ordinary skill in the art. Human IL-11Rα sequences are thus available from the NCBI website at http:I/www.ncbi.nlm.nih.gov/entrez under the nucleotide accession number Z38102, the content of which is herein incorporated by reference. IL-11Rα sequences from animal yet non-human origin, such as from mouse and rat, are also available from the NCBI website at http://www.ncbi.nlm.nih.gov/entrez (for mouse and rat IL-11Rα sequences, see under the respective nucleotide accession numbers X98519 and AF347936, the contents of which are herein incorporated by reference).

As a matrix to assay for binding to IL-11Rα, soluble IL-11Rα, e.g. the human IL-11Rα-IL-2 fusion protein which is described in Blanc et al. 2000, can be used (Blanc et al. (2000) Monoclonal antibodies against the human interleukin-11 receptor alpha-chain (IL-11Ralpha) and their use in studies of human mononuclear cells. J. Immunol. Methods 241, 43-59, the content of which is herein incorporated by reference). Murine IL11-11Ralpha is available from R&D Sytems (http://www.RnDSystems.com).

Wild-type IL-11 nucleotide and amino acid sequences are available from standard sequence databanks known to the person of ordinary skill in the art: human wild-type IL-11 sequence is described on the NCBI website at http://www.ncbi.nlm.nih.gov/entrez under the nucleotide accession number AY207429, the content of which is herein incorporated by reference. Human wild-type nucleotide and amino acid sequences are also shown printed from said site on the enclosed FIG. 1 (SEQ ID NO:73=AY207429 IL-11 nucleotide sequence; SEQ ID NO: 1=human wild-type IL-11 amino acid sequence). A wild-type hIL-11 cDNA sequence is also available from Accession Number NM57765 from the above-mentioned NCBI website.

Wild-type IL-11 sequences from animal yet non-human sources are also available from the above-mentioned NCBI website, such as e.g. mouse and rat IL-11 (nucleotide accession number NM 008350 and NM 133519, respectively).

The replacement of said at least two amino acids can be achieved by any standard procedure known to the person of ordinary skilled in the art. It may e.g. involve mutation by inverse PCR amplification as described in Stemmer W. P. and Morris S. K. 1992 [Stemmer and Morris (1992) Enzymatic inverse PCR: a restriction site independent, single-fragment method for high-efficiency, site-directed mutagenesis. Biotechniques 13, 214-220], of which content is herein incorporated by reference. The choice of appropriate primers is made by making use of the common knowledge in the field applied to the design of oligonucleotides which have such a sequence that they can have the function of a primer for a given IL-11 template sequence, while having the ability to introduce at least two point mutations in the amplicon with respect to the template sequence (see Stemmer and Morris 1992, cited supra).

An illustrative procedure of such an IL-11 mutagenesis is described in example 1 below.

The production of the mutein can be achieved by any conventional procedure known to the person of ordinary skill in the art of producing proteins in general, and of producing wild-type IL-11 in particular. It may e.g. comprise the production of a plasmid comprising a sequence coding for the mutein (for the construction of a plasmid, see Wang, X. M., Wilkin, J. M., Boisteau, O., Harmegnies, D., Blanc, C., Vandenbussche, P., Montero-Julian, F. A., Jacques, Y. and Content, J. (2002) Engineering and use of 32P-labelled human recombinant interleukin-11 for receptor binding studies. Eur. J. Biochem. 269, 61-68, the content of which is herein incorporated by reference), and transforming a host cell such as E. coli with this plasmid so that the the mutein is expressed by the transformed cells, from which it can be recovered and isolated. An illustrative procedure of mutein production is described in example 1 below.

Non-hydrophobic amino acids (e.g. hydrophilic amino acids) have a side chain that is electrically charged, or that is an uncharged yet polar chain. They notably comprise:
Cystein (symbol=C or Cys),
Tyrosine (symbol=Y or Tyr),
Histidine (symbol=H or His),
Lysine (symbol=K or Lys),
Arginine (symbol=R or Arg),
Glutamine (symbol=Q or Gln),
Asparagine (symbol=N or Asn),
Glutamic acid (symbol=E or Glu),
Aspartic acid (symbol=D or Asp),
Serine (symbol=S or Ser),
Threonine (symbol=T or Thr).

Hydrophobic amino acids have a side chain that is non-polar and uncharged. They notably comprise:
Valine (symbol=V or Val),
Alanine (symbol A or Ala),
Proline (symbol P or Pro),
Leucine (symbol=L or Leu),
Isoleucine (symbol=I or Ile),
Methionine (symbol=M or Met),
Tryptophan (symbol=W or Trp),
Phenylalanine (symbol=F or Phe).

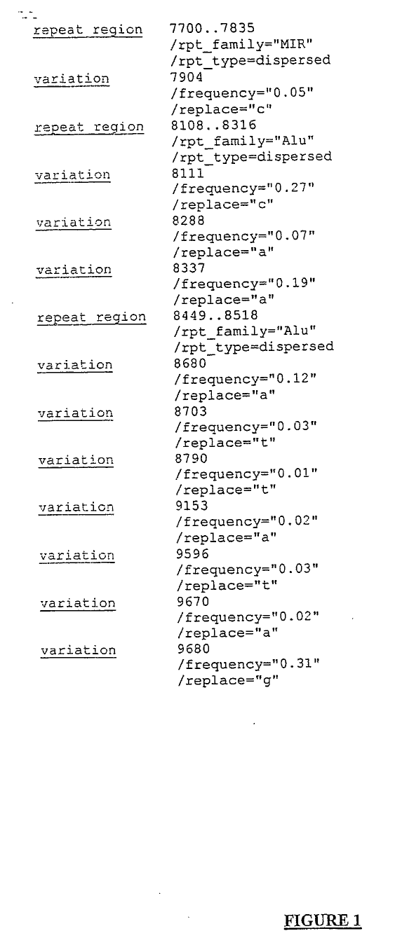

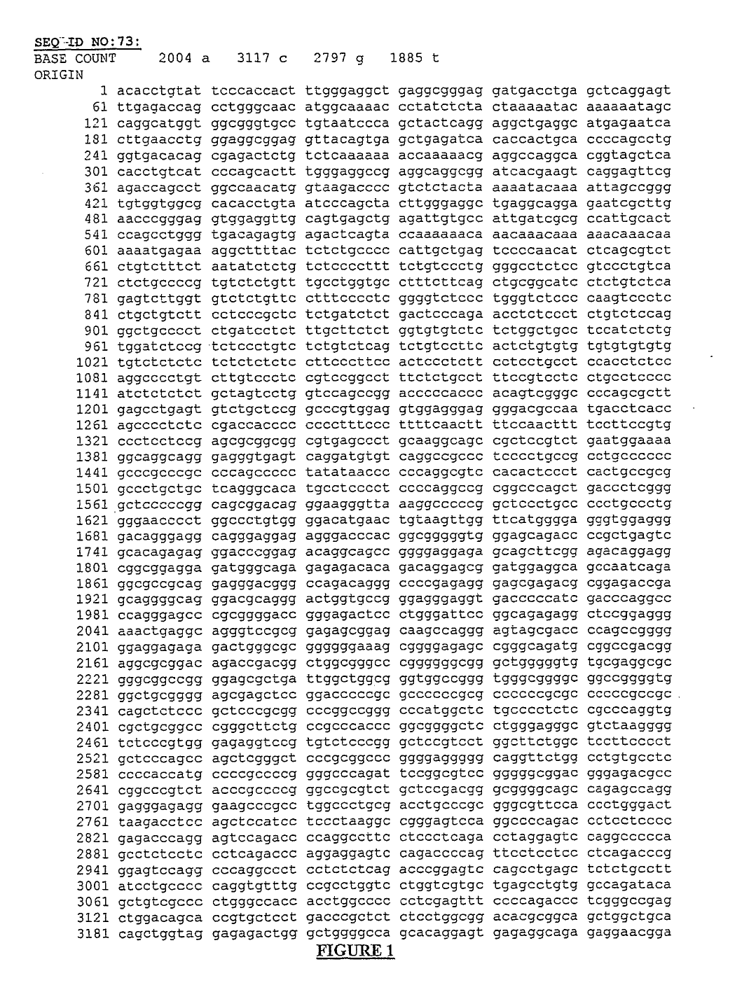

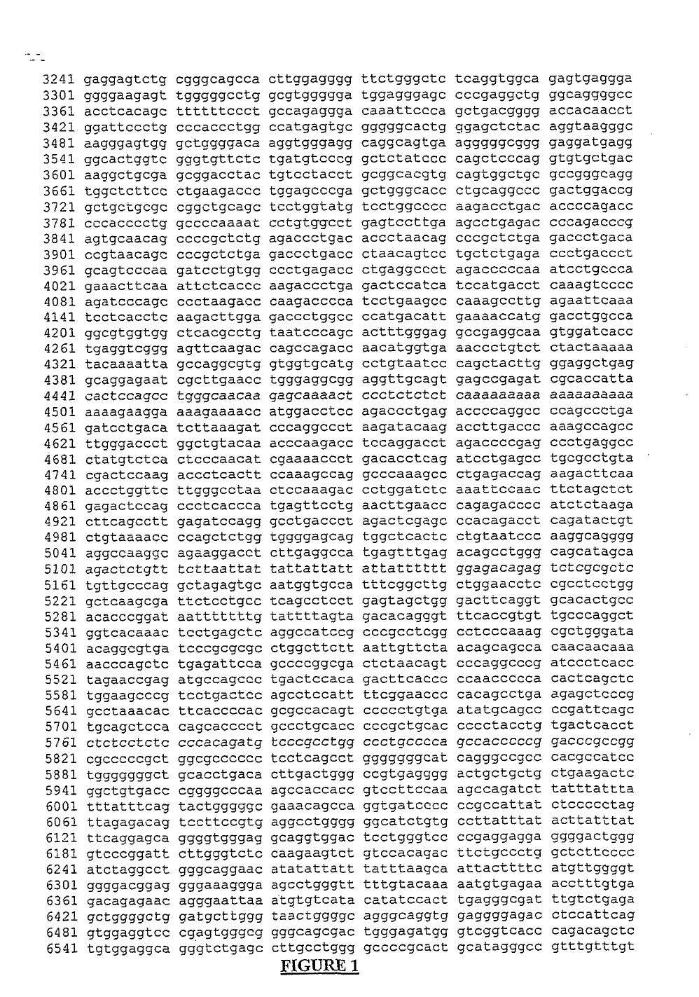

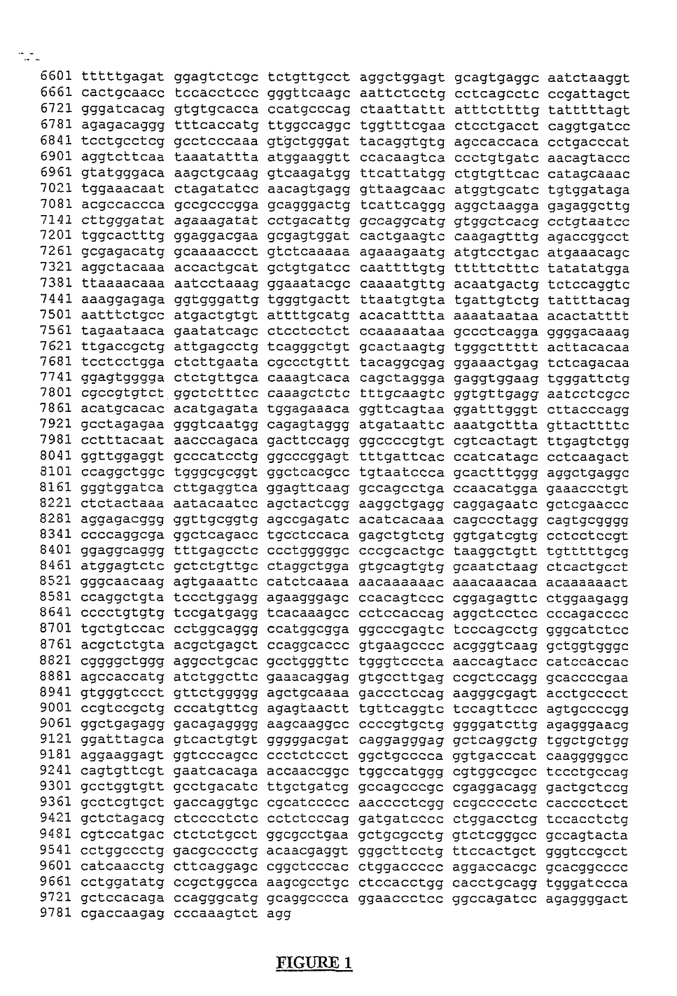

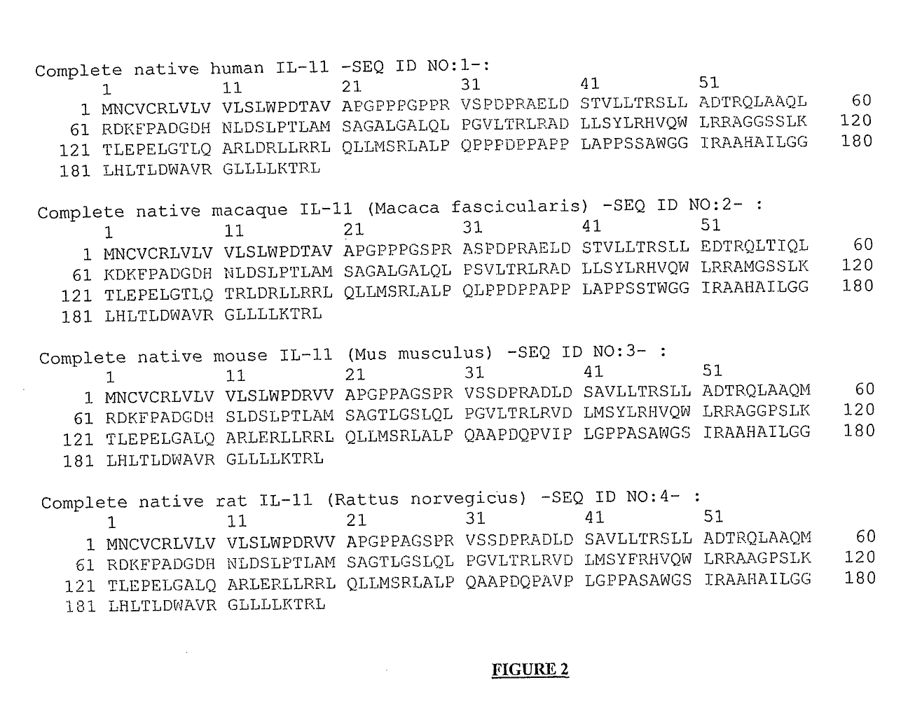

Human IL-11 site I is composed of a hydrophobic cluster which comprises a limited number of hydrophilic amino acids: these site I hydrophilic amino acids notably comprise H in position 182 and D in position 186 (see SEQ ID NO:1 on FIG. 1 and on FIG. 2).

In accordance with the present invention, histidine (H) in position 182 and aspartic acid (D) in position 186 are most preferred as wild-type hIL-11 mutation targets to be replaced by hydrophobic amino acids.

Similarity in terms of sterical hindrance, structure and/or size may in choosing those hydrophobic amino acids which are more appropriate to replace said H and D targets. The most preferred hydrophobic amino acids for replacing IL-11 site I hydrophilic amino acids comprise valine (V) and alanine (A).

The mutein obtained by replacement of H182 by V and of D186 by A has proven to be an IL-11 hyperagonist: compared to wild-type hIL-11, it has a three-fold increased affinity for IL-11Rα, while still retaining the ability to recruit gp130; it is 60 to 400 fold more active on the proliferation of the murine hybridoma cell line 7TD1, and the mutein reaches an in vivo in vivo radioprotection iso-effect at a ten-fold lower dose than the wild-type IL-11 (ten fold less mutein than wild-type IL-11 is needed to achieve the same in vivo radioprotection effect); see examples 1 and 2 below.

In macaque, mouse and rat wild-type IL-11, those hydrophilic amino acids which at present are known to belong to site I are also H182 and D186.

The N-terminal of wild-type IL-11 protein begins with a signal peptide of 21 amino acids, directly followed by a proline-rich region of 13 amino acids. These first 34 N-terminal amino acids are not necessary to IL-11 biological activity: they can therefore be deleted. FIG. 3 shows the wild-type human, macaque, mouse and rat IL-11 sequences respectively deleted from their first 34 N-terminal amino acids (SEQ ID NO: 5-8, respectively).

The present invention thus provides IL-11 muteins, the sequence of which comprises a sequence which is derivable from a wild-type IL-11 by replacement of the hydrophilic amino acids in positions 182 and 186 (positions computed by reference to the complete wild-type sequence) by X₁ and X₂ respectively, X₁ and X₂ being chosen from the group comprising:
Valine (symbol=V or Val),
Alanine (symbol=A or Ala),
Proline (symbol=P or Pro),
Leucine (symbol=L or Leu),
Isoleucine (symbol=I or Ile),
Phenylalanine (symbol=F or Phe),
Methionine (symbol=M or Met), and
Tryptophan (symbol=W or Trp),
   and optionally by deletion of a N-terminal portion that does not exceed the first 34 N-terminal amino acids.

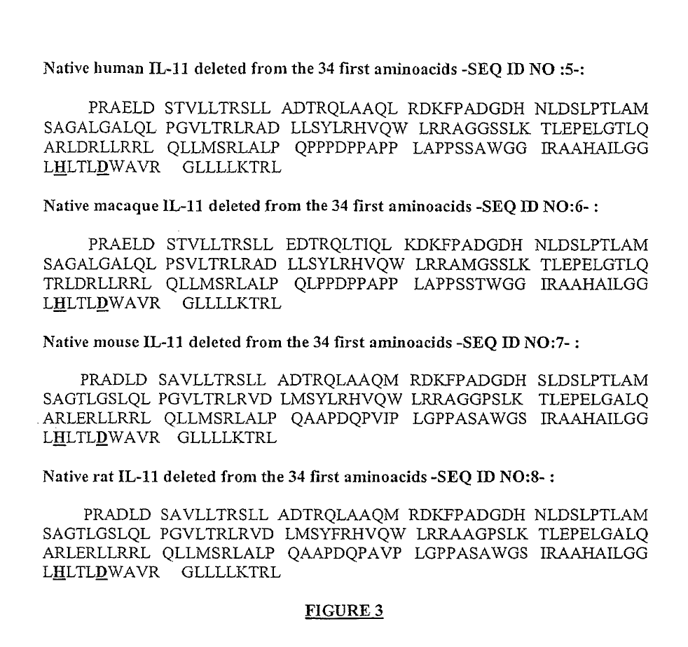

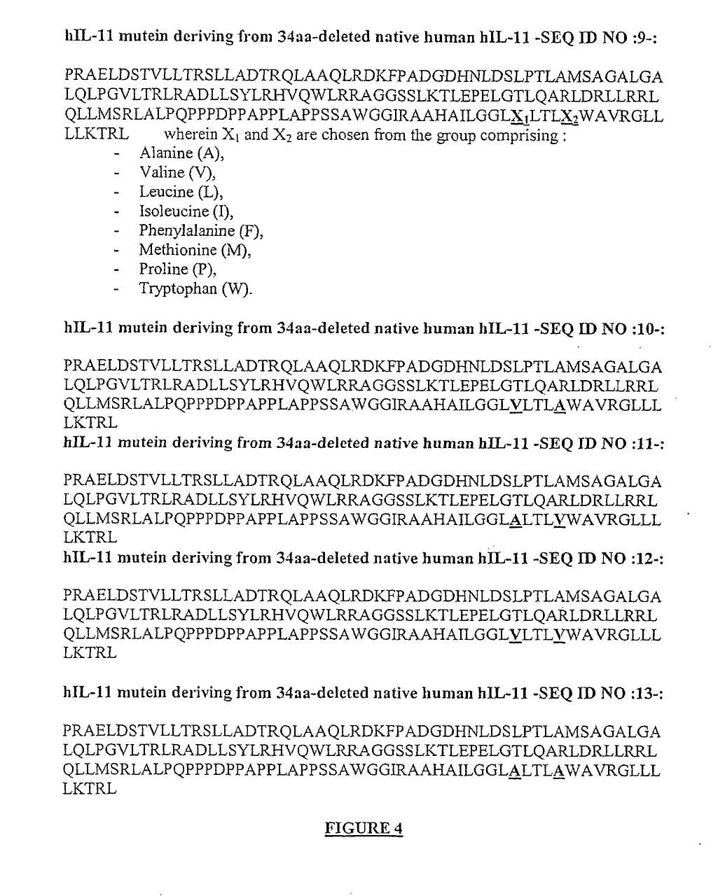

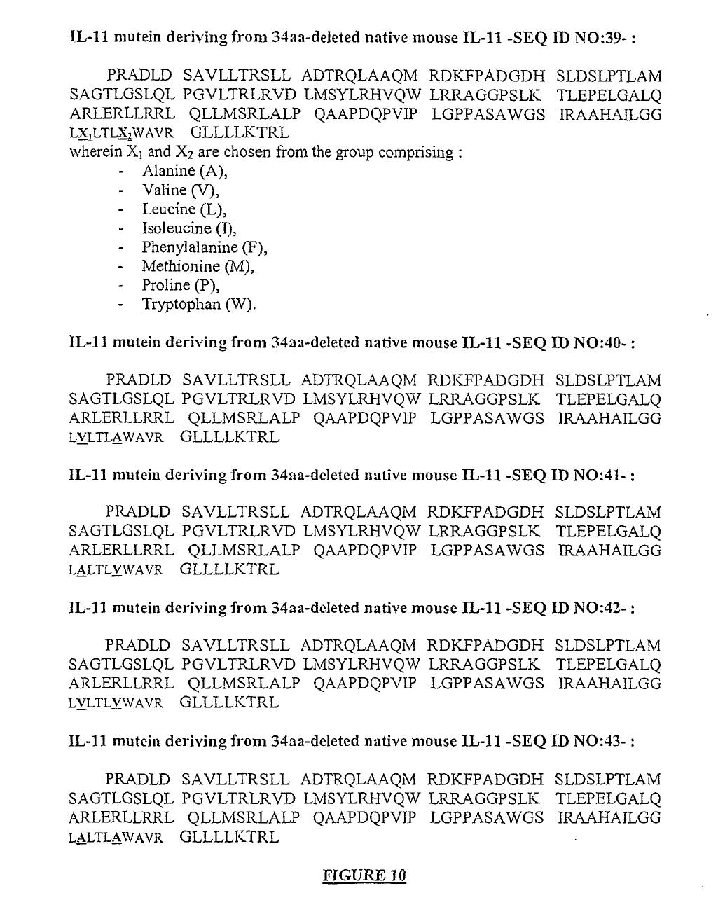

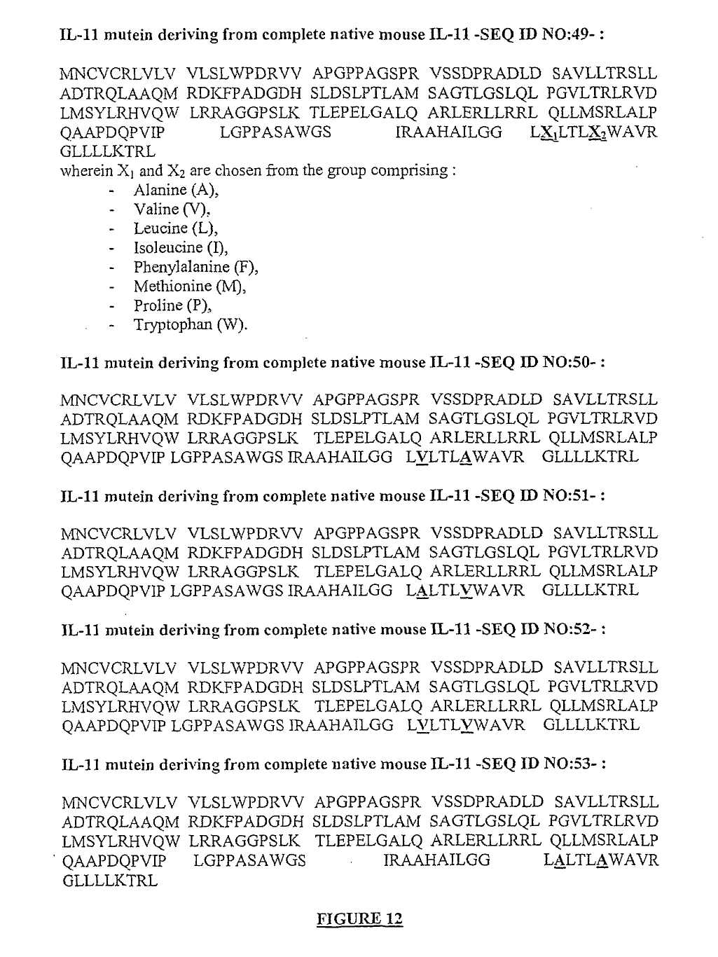

As the first 34 N-terminal amino acids are not necessary to IL-11 biological activity, a N-terminal portion that does not exceed the first 34 N-terminal amino acids can indeed be deleted without substantially altering the functions of the muteins of the invention. The present invention thus relates to IL-11 muteins, the sequence of which comprises a sequence chosen from the group comprising SEQ ID NO:9, SEQ ID NO:24, SEQ ID NO:39, SEQ ID NO:54 shown on FIGS. 4, 7, 10, 13, respectively (IL-11 muteins which derives from 34aa-deleted wild-type IL-11 from human, macaque, mouse and rat origin, respectively).

The-present invention also encompasses those equivalent IL-11 muteins which comprise a sequence of at least 80%, preferably at least 90% identity with the above-mentioned SEQ ID NO:9, SEQ ID NO:24, SEQ ID NO:39, or SEQ ID NO:54, provided that X₁ and X₂ are as above-defined (i.e. hydrophobic amino acids), and that the resulting protein has retained the ability of wild-type IL-11 to induce proliferation of an IL-11 dependent cell line, such as e.g. the 7TD1 murine hybridoma cell line.

Illustrative and useful muteins of the invention comprise those for which X1 and X2 are V or A.

The present invention therefore more particularly encompasses those IL-11 muteins which comprise a sequence corresponding to a wild-type IL-11 deleted from those N-terminal amino acids which are not necessary to its biological activity, wherein the amino acids in positions 182 and 186 have been replaced by V and A, A and V, V and V, or A and A, respectively.

The present invention thus relates to those IL-11 muteins which comprise a sequence of SEQ ID NO:9, SEQ ID NO:24, SEQ ID NO:39, or SEQ ID NO:54, wherein X₁=V and X₂=A, i.e. to those IL-11 muteins which comprise a sequence of SEQ ID NO:10 (deriving from human IL-11), of SEQ ID NO:25 (deriving from macaque IL-11), of SEQ ID NO:40 (deriving from mouse IL-11), or of SEQ ID NO:55 (deriving from rat IL-11). These SEQ ID are shown on FIGS. 4, 7, 10 and 13, respectively.

The present invention also relates to those IL-11 muteins which comprise a sequence of SEQ ID NO:9, SEQ ID NO:24, SEQ ID NO:39, or SEQ ID NO:54, wherein X₁=A and X₂=V, i.e. to those IL-11 muteins which comprise a sequence of SEQ ID NO:11 (deriving from human IL-11), of SEQ ID NO:26 (deriving from macaque IL-11), of SEQ ID NO:41 (deriving from mouse IL-11), or of SEQ ID NO:56 (deriving from rat IL-11). These SEQ ID are shown on FIGS. 4, 7, 10 and 13, respectively.

The present invention also relates to those IL-11 muteins which comprise a sequence of SEQ ID NO:9, SEQ ID NO:24, SEQ ID NO:39, or SEQ ID NO:54, wherein X₁=V and X₂=V, i.e. to those IL-11 muteins which comprise a sequence of SEQ ID NO:12 (deriving from human IL-11), of SEQ ID NO:27 (deriving from macaque IL-11), of SEQ ID NO:42 (deriving from mouse IL-11 or of SEQ ID NO:57 (deriving from rat IL-11). These SEQ ID are shown on FIGS. 4, 7, 10 and 13, respectively.

The present invention also relates to those IL-11 muteins which comprise a sequence of SEQ ID NO:9, SEQ ID NO:24, SEQ ID NO:39, or SEQ ID NO:54, wherein X₁=A and X₂=A, i.e. to those IL-11 muteins which comprise a sequence of SEQ ID NO:13 (deriving from human IL-11), of SEQ ID NO:28 (deriving from macaque IL-11), of SEQ ID NO:43 (deriving from mouse IL-11), or of SEQ ID NO:58 (deriving from rat IL-11). These SEQ ID are shown on FIGS. 4, 7, 10 and 13, respectively.

Illustrative and useful IL-11 muteins of the invention thus comprises those IL-11 muteins which derive from a wild-type IL-11 by deletion of the signal peptide (first 21 N-terminal amino acids), and by replacement of the amino acids in positions 182 and 186 (positions computed by reference to the complete wild-type IL-11 by the hydrophobic X₁ and X₂ amino acids as above-defined.

The present invention thus encompasses those IL-11 muteins, the sequence of which comprises or consists in a sequence of SEQ ID NO:14, SEQ ID NO:29, SEQ ID NO:44 or SEQ ID NO:59, wherein X₁ and X₂ are as above-defined. These SEQ ID are shown on FIGS. 5, 8, 11, and 14, respectively.

The sequence of SEQ ID NO:14 corresponds to the human wild-type IL-11 wherein the amino acids in positions 182 and 186 have been replaced by X₁ and X₂, and wherein the first 21 N-terminal amino acids have been deleted (see FIG. 5).

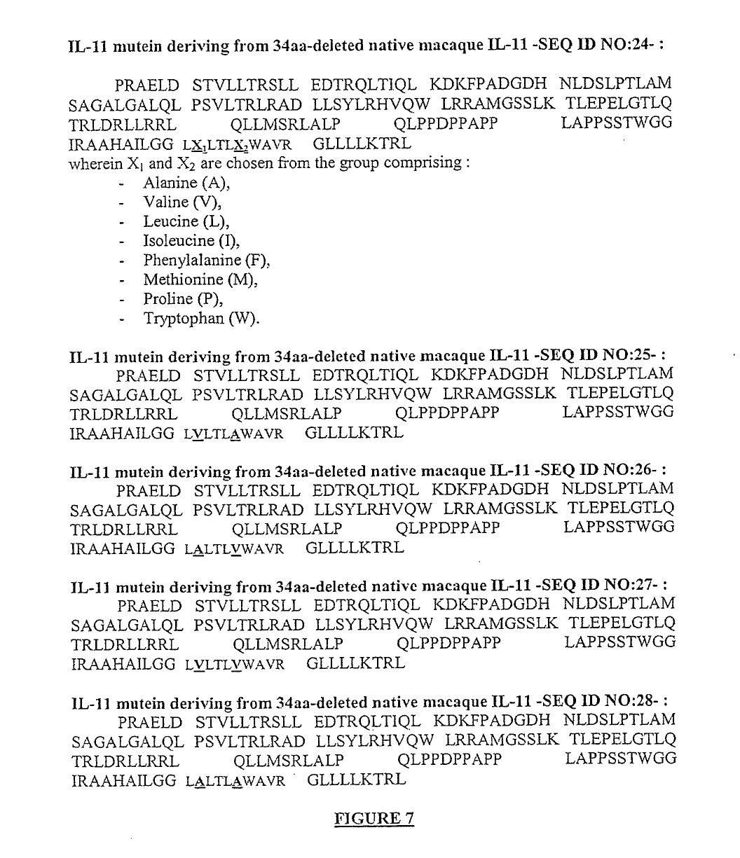

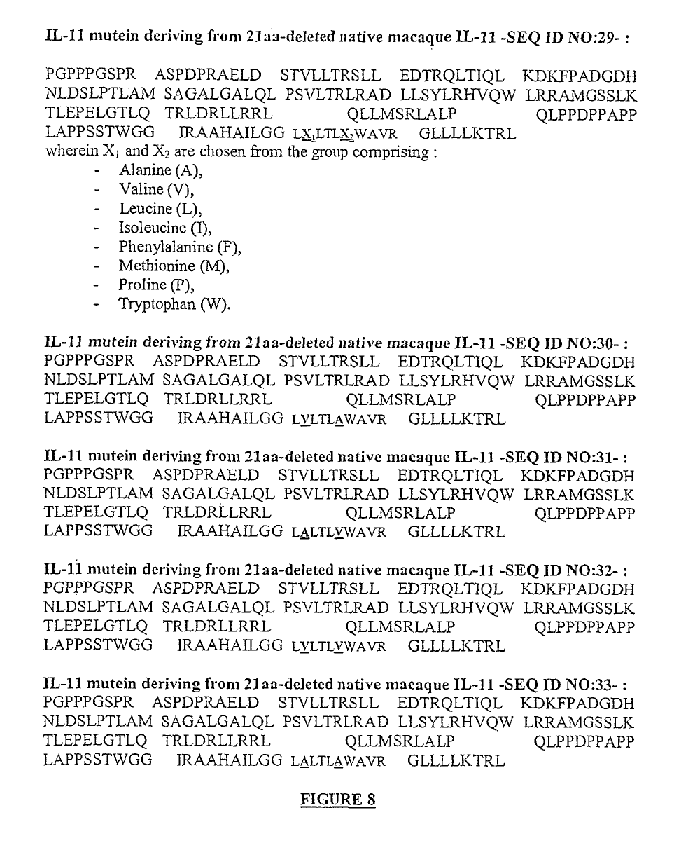

The sequence of SEQ ID NO:29 corresponds to the macaque wild-type IL-11 wherein the amino acids in positions 182 and 186 have been replaced by X₁ and X₂, and wherein the first 21 N-terminal amino acids have been deleted (see FIG. 8).

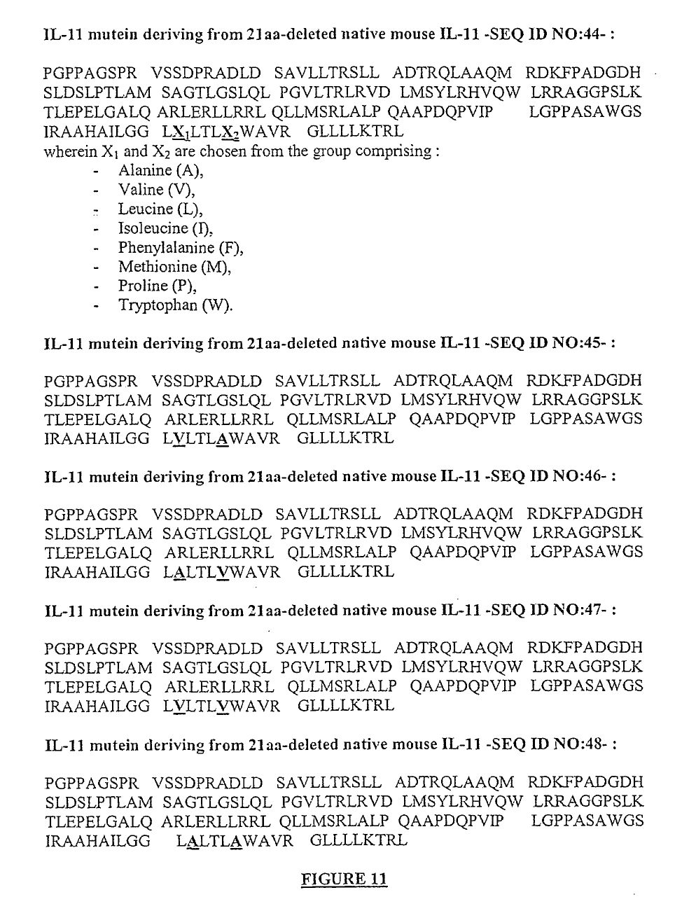

The sequence of SEQ ID NO:44 corresponds to the mouse wild-type IL-11 wherein the amino acids in positions 182 and 186 have been replaced by X₁ and X₂, and wherein the first 21 N-terminal amino acids have been deleted (see FIG. 11).

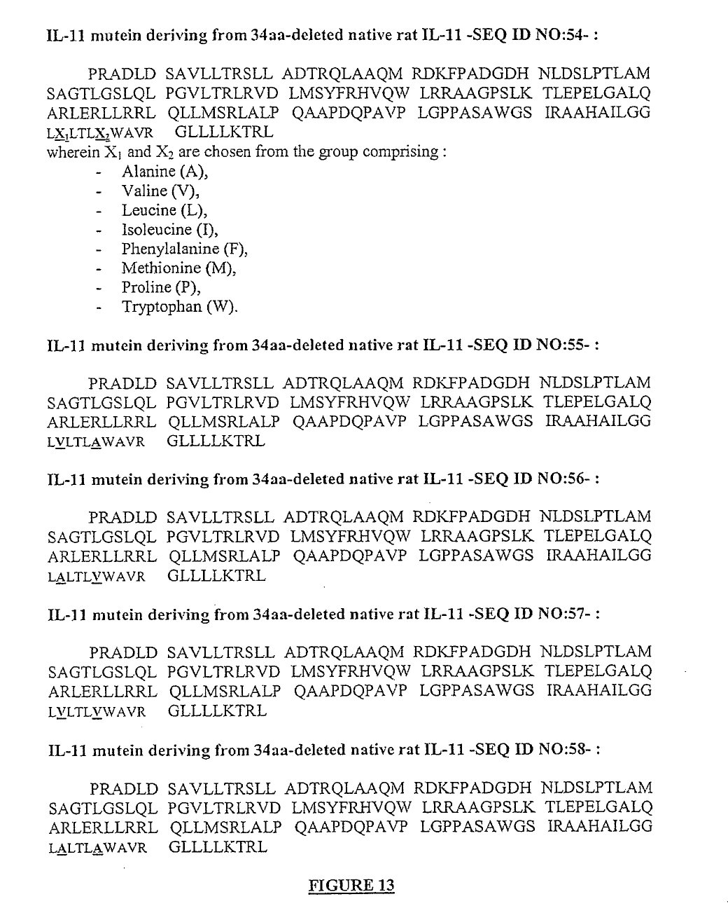

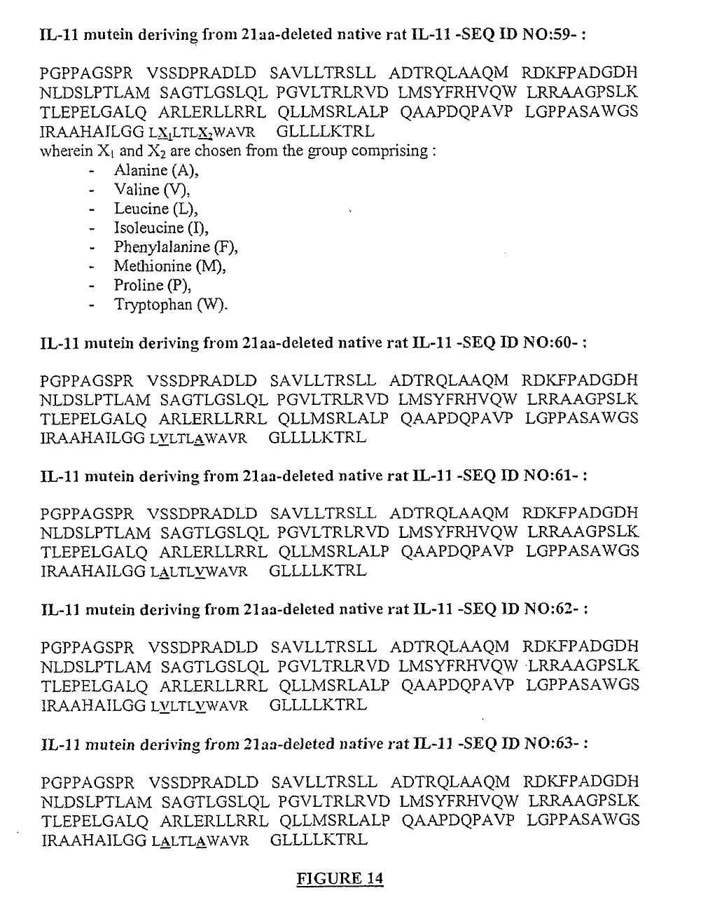

The sequence of SEQ ID NO:59 corresponds to the rat wild-type IL-11 wherein the amino acids in positions 182 and 186 have been replaced by X₁ and X₂, and wherein the first 21 N-terminal amino acids have been deleted (see FIG. 14).

When X₁=V and X₂=A in SEQ ID NO:14, SEQ ID NO:29, SEQ ID NO:44, SEQ ID NO:59, respectively, the muteins of the invention comprise or consist in a sequence of SEQ ID NO:15, SEQ ID NO:30, SEQ ID NO:45, SEQ ID NO:60, respectively (shown on FIGS. 5, 8, 11, 14, respectively).

When X₁=A and X₂=V in SEQ ID NO:14, SEQ ID NO:29, SEQ ID NO:44, SEQ ID NO:59, respectively, the muteins of the invention comprise or consist in a sequence of SEQ ID NO:16, SEQ ID NO:31, SEQ ID NO:46, SEQ ID NO:61, respectively (shown on FIGS. 5, 8, 11, 14, respectively).

When X₁=V and X₂=V in SEQ ID NO:14, SEQ ID NO:29, SEQ ID NO:44, SEQ ID NO:59, respectively, the muteins of the invention comprise or consist in a sequence of SEQ ID NO:17, SEQ ID NO:32, SEQ ID NO:47, SEQ ID NO:62, respectively (shown on FIGS. 5, 8, 11, 14, respectively).

When X₁=A and X₂=A in SEQ ID NO:14, SEQ ID NO:29, SEQ ID NO:44, SEQ ID NO:59, respectively, the muteins of the invention comprise or consist in a sequence of SEQ ID NO:18, SEQ ID NO:33, SEQ ID NO:48, SEQ ID NO:63, respectively (shown on FIGS. 5, 8, 11, 14, respectively).

The muteins of the invention which comprise or consist in a sequence of SEQ ID NO:15-18, SEQ ID NO:30-33, SEQ ID NO:45-48, SEQ ID NO:60-63, respectively are preferred muteins of the invention. Those muteins of the invention which comprise or consist in a sequence of SEQ ID NO:15, SEQ ID NO:30, SEQ ID NO:45, SEQ ID NO:60, respectively are most preferred.

Illustrative and useful IL-11 muteins of the invention are also those muteins of the invention that derive from complete wild-type IL-11 by replacement of the amino acids in positions 182 and 186 by the hydrophobic X₁ and X₂ amino acids as above defined. Such illustrative and useful IL-11 muteins thus comprise those that comprise or consist in a sequence of SEQ ID NO:19, SEQ ID NO:34, SEQ ID NO:49, or SEQ ID NO:64, wherein X₁ and X₂ are as above defined.

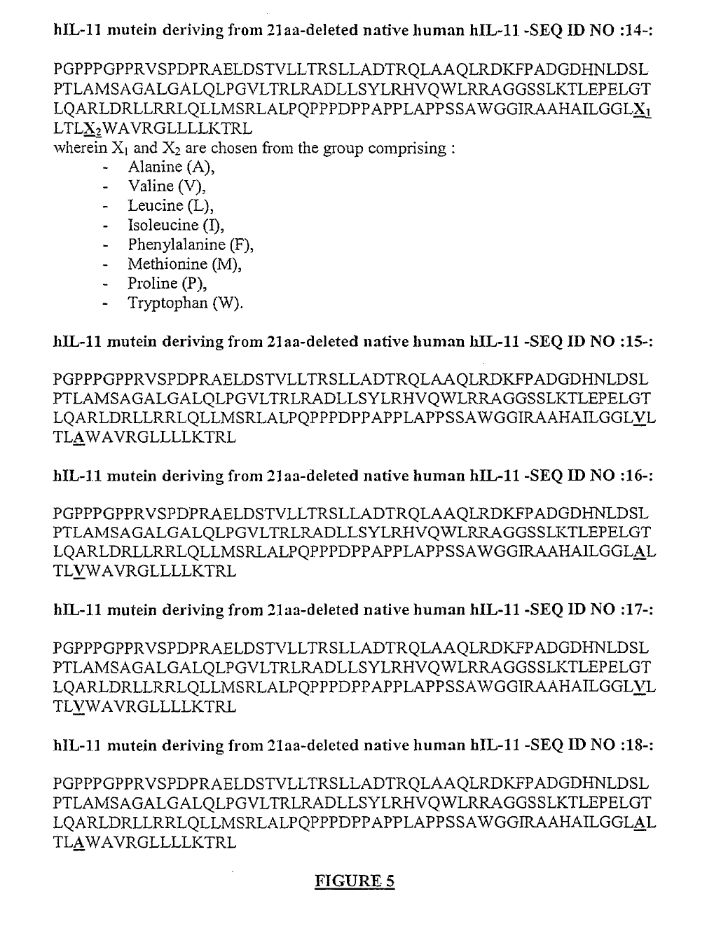

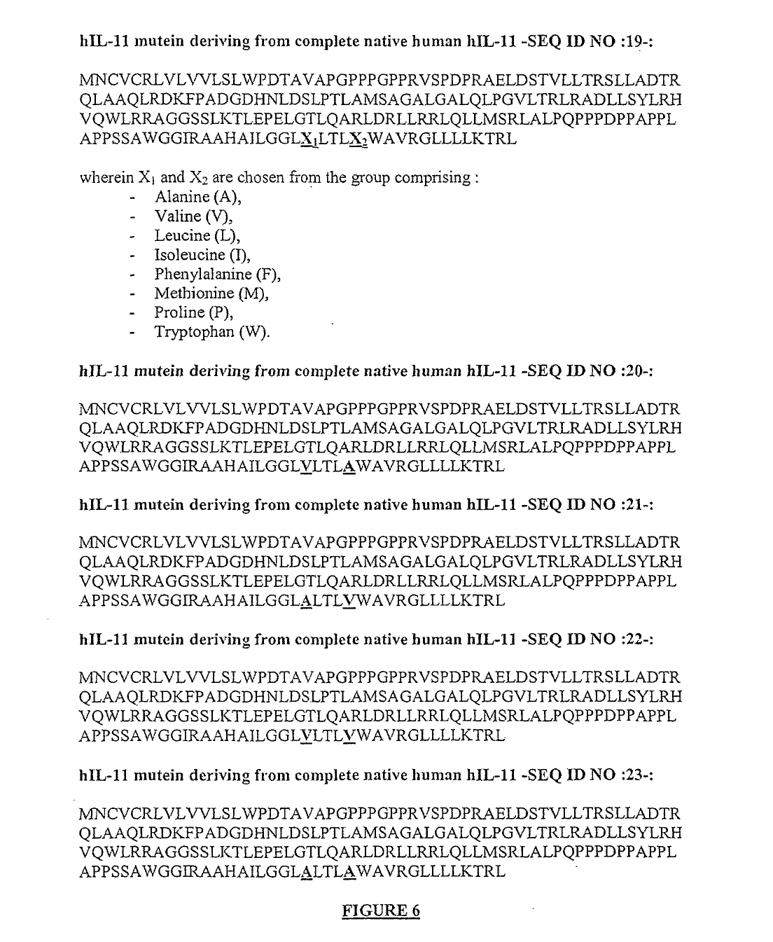

The sequence of SEQ ID NO:19 corresponds to human complete wild-type IL-11 wherein H182 and D186 have both been replaced by X₁ and X₂ as above defined. It is shown on FIG. 6.

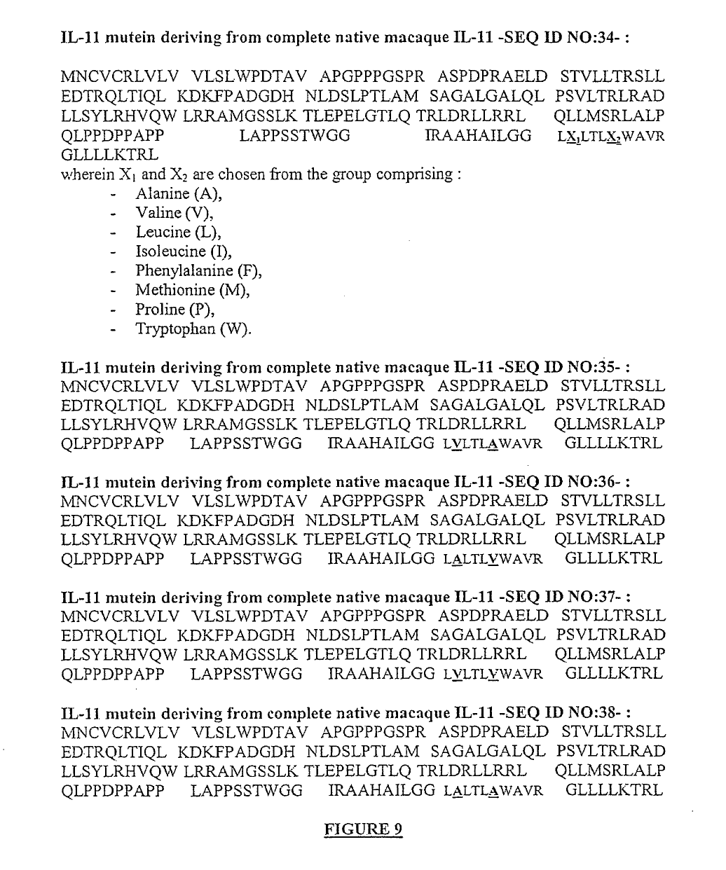

The sequence of SEQ ID NO:34 corresponds to macaque complete wild-type IL-11 wherein H182 and D186 have both been replaced by X₁ and X₂ as above defined. It is shown on FIG. 9.

The sequence of SEQ ID NO:49 corresponds to mouse complete wild-type IL-11 wherein H182 and D186 have both been replaced by X₁ and X₂ as above defined. It is shown on FIG. 12.

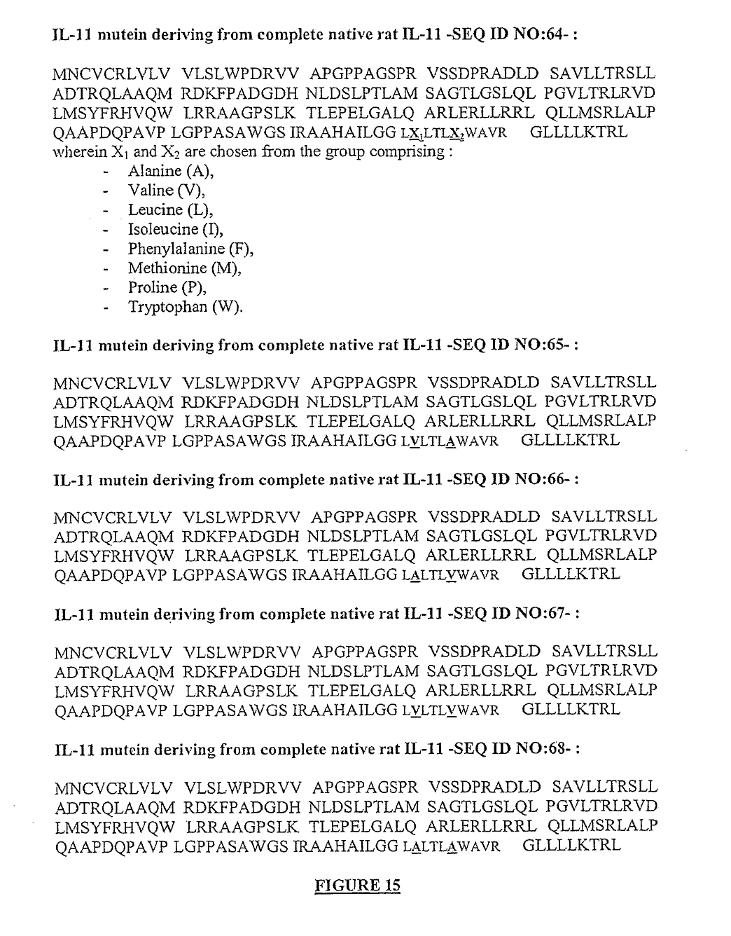

The sequence of SEQ ID NO:64 corresponds to rat complete wild-type IL-11 wherein H182 and D186 have both been replaced by X₁ and X₂ as above defined. It is shown on FIG. 15.

When X₁=V and X₂=A in SEQ ID NO:19, SEQ ID NO:34, SEQ ID NO:49, SEQ ID NO:64, respectively, the muteins of the invention comprise or consist in a sequence of SEQ ID NO:20, SEQ ID NO:35, SEQ ID NO:50, SEQ ID NO:65, respectively (shown on FIGS. 6, 9, 12, 15, respectively).

When X₁=A and X₂=V in SEQ ID NO:19, SEQ ID NO:34, SEQ ID NO:49, SEQ ID NO:64, respectively, the muteins of the invention comprise or consist in a sequence of SEQ ID NO:21, SEQ ID NO:36, SEQ ID NO:51, SEQ ID NO:66, respectively (shown on FIGS. 6, 9, 12, 15, respectively).

When X₁=V and X₂=V in SEQ ID NO:19, SEQ ID NO:34, SEQ ID NO:49, SEQ ID NO:64, respectively, the muteins of the invention comprise or consist in a sequence of SEQ ID NO:22, SEQ ID NO:37, SEQ ID NO:52, SEQ ID NO:67, respectively (shown on FIGS. 6, 9, 12, 15, respectively).

When X₁=A and X₂=A in SEQ ID NO:19, SEQ ID NO:34, SEQ ID NO:49, SEQ ID NO:64, respectively, the muteins of the invention comprise or consist in a sequence of SEQ ID NO:23, SEQ ID NO:38, SEQ ID NO:53, SEQ ID NO:68, respectively (shown on FIGS. 6,9, 12, 15, respectively).

The muteins of the invention which comprise or consist in a sequence of SEQ ID NO:20-23, SEQ ID NO:35-38, SEQ ID NO:50-53, SEQ ID NO:65-68, respectively are preferred muteins of the invention. Those muteins of the invention which comprise or consist in a sequence of SEQ ID NO:20, SEQ ID NO:35, SEQ ID NO:50, SEQ ID NO:65, respectively are most preferred.

The present invention also encompasses any nucleic acid, such as DNA or RNA, coding for a mutein of the invention.

It notably encompasses any nucleic acid, such as DNA, which comprises the joined CDS (coding sequences) of a wild-type IL-11 appropriately mutated in accordance with the present invention.

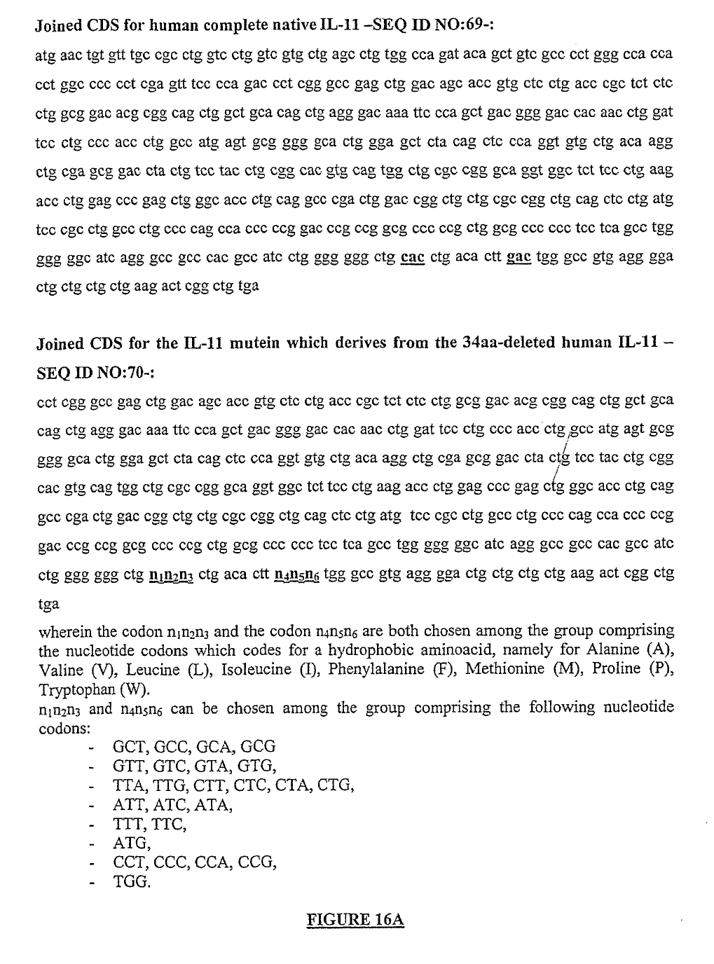

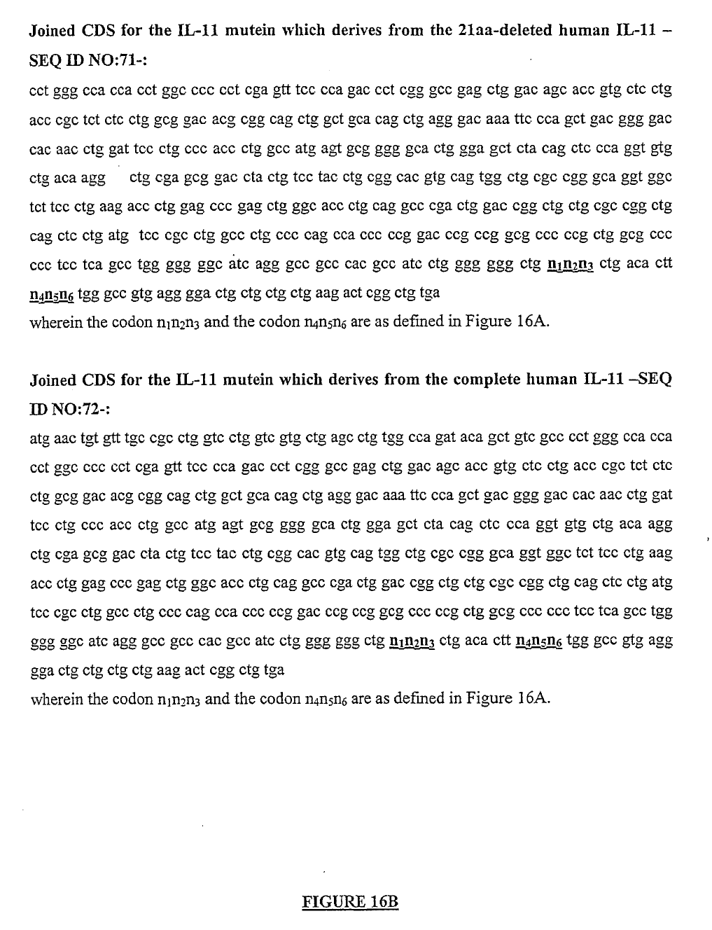

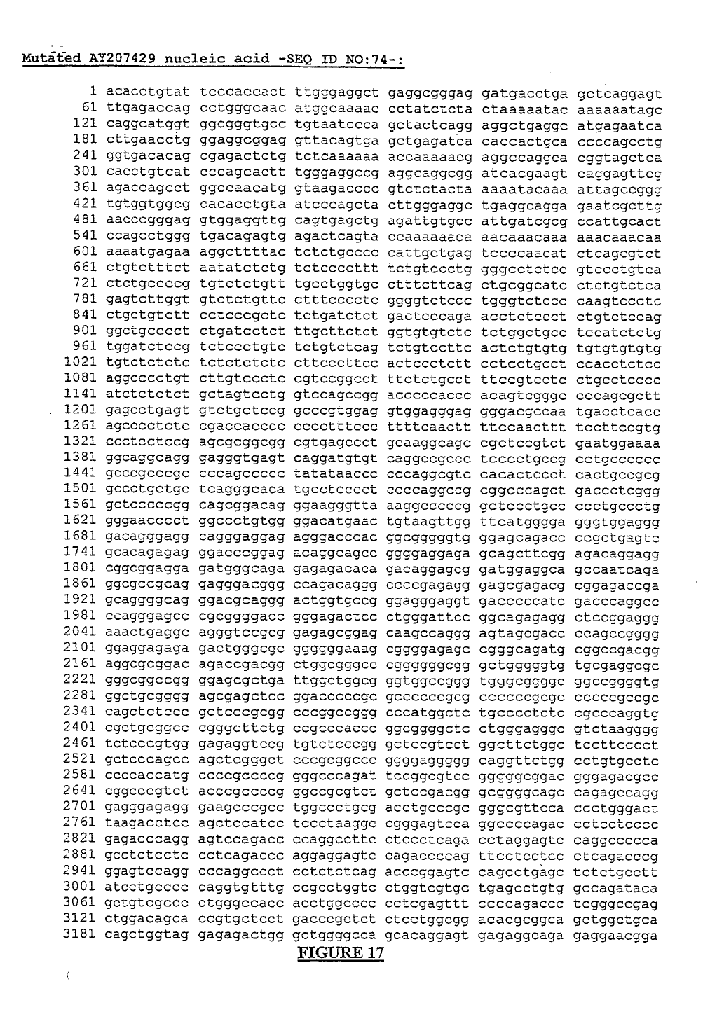

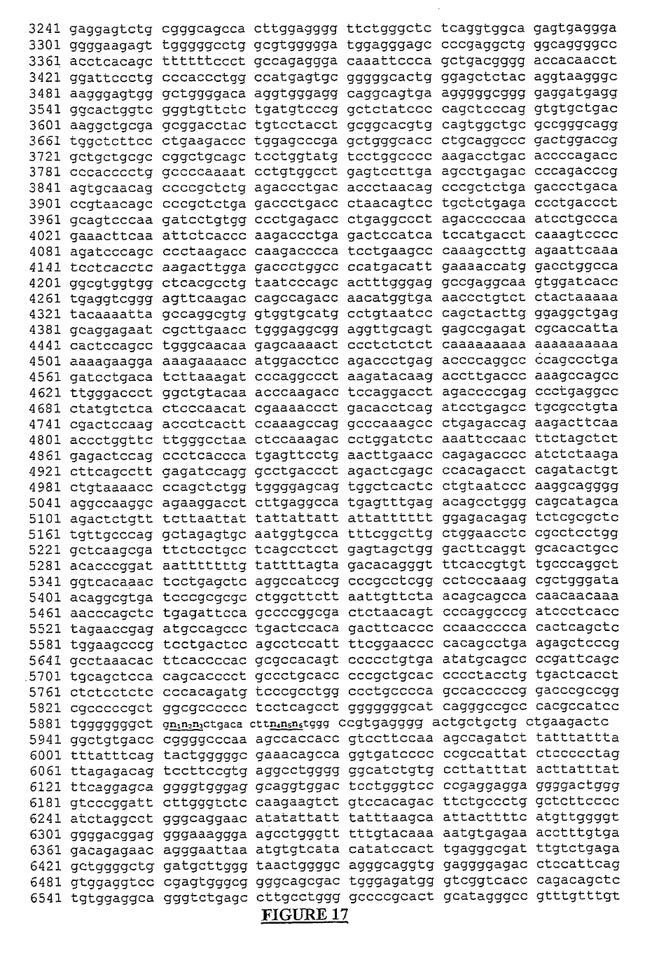

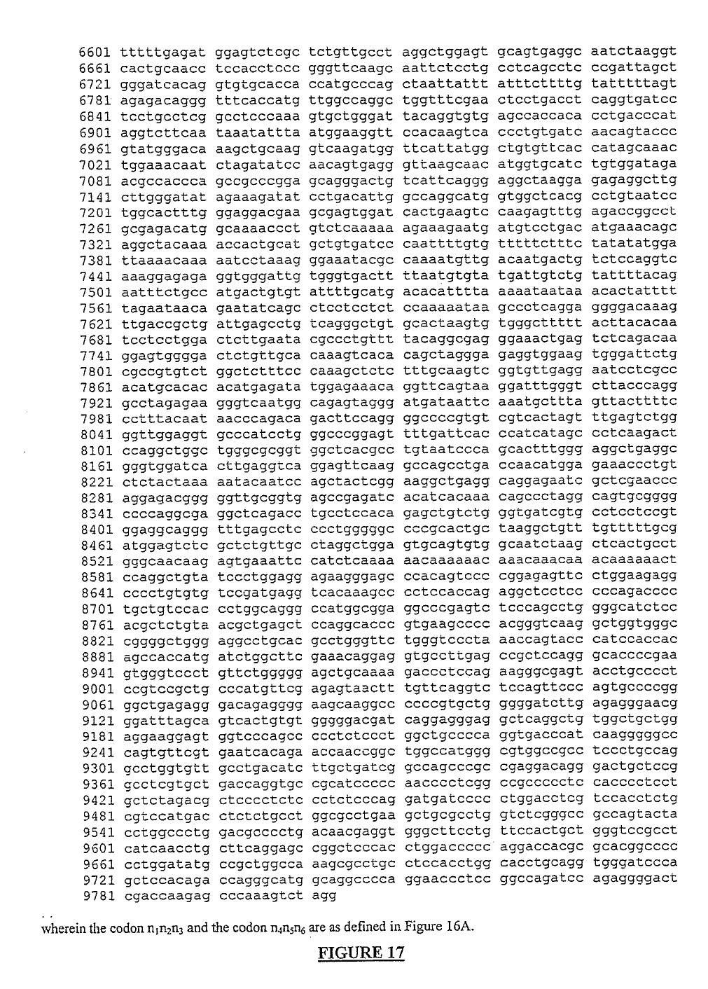

The joined CDS sequence of human wild-type IL-11 are shown as SEQ ID NO:69 on FIG. 16A (codon CAC coding for H182, and codon GAC coding for D186 are underlined).

Appropriate mutations in accordance with the present invention comprise replacing said cac and gac wild-type codons by codon n₁n₂n₃ and n₄n₅n₆ respectively, wherein both n₁n₂n₃ and n₄n₅n₆ code for hydrophobic amino acids, i.e. the above-defined X1 and X2 amino acids.

Accordingly, n₁n₂n₃ and n₄n₅n₆ are both chosen from the group comprising the nucleotide codons which code for Valine (symbol=V or Val), Alanine (symbol=A or Ala), Proline (symbol=P or Pro), Leucine (symbol=L or Leu), Isoleucine (symbol=I or Ile), Phenylalanine (symbol=F or Phe), Methionine (symbol=M or Met), and Tryptophan (symbol=W or Trp). It follows that, having taken into account the degeneracy of the genetic code, n₁n₂n₃ and n₄n₅n₆ are both chosen from the group comprising the following codons:

GCT, GCC, GCA, GCG,
GTT, GTC, GTA, GTG,
TTA, TTG, CTT, CTC, CTA, CTG,
ATT, ATC, ATA,
TTT, TTC,
ATG,
CCT, CCC, CCA, CCG,
TGG.

The present invention thus notably encompasses any nucleic acid (e.g. DNA) which comprises the sequence of SEQ ID NO:72 shown on FIG. 16B, wherein n₁n₂n₃ and n₄n₅n₆ are as above-defined.

When, in accordance with the present invention, the wild-type IL-11 has been deleted from its first 21 N-terminal amino acid (see SEQ ID NO:14-18 on FIG. 5), or from its first 34 N-terminal amino acid (see SEQ ID NO:9-13 on FIG. 4), the corresponding joined CDS sequence is deleted from the corresponding codons.

The present invention thus encompasses any nucleic acid (e.g. DNA) which comprises the sequence of SEQ ID NO:71 or of SEQ ID NO:70 shown on FIG. 16B and 16A, respectively, wherein n₁n₂n₃ and n₄n₅n₆ are as above-defined.

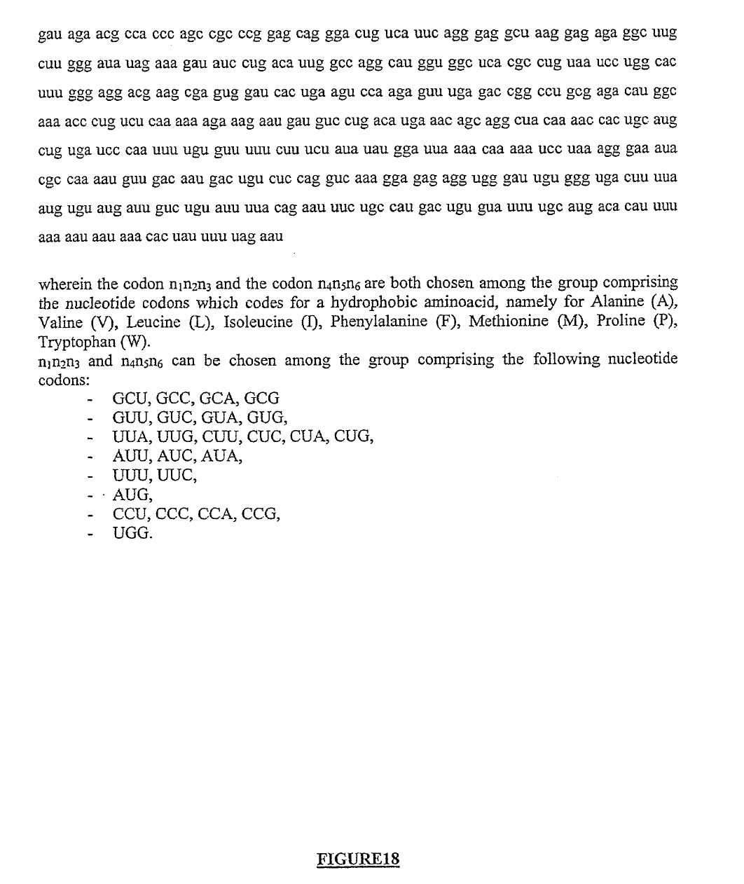

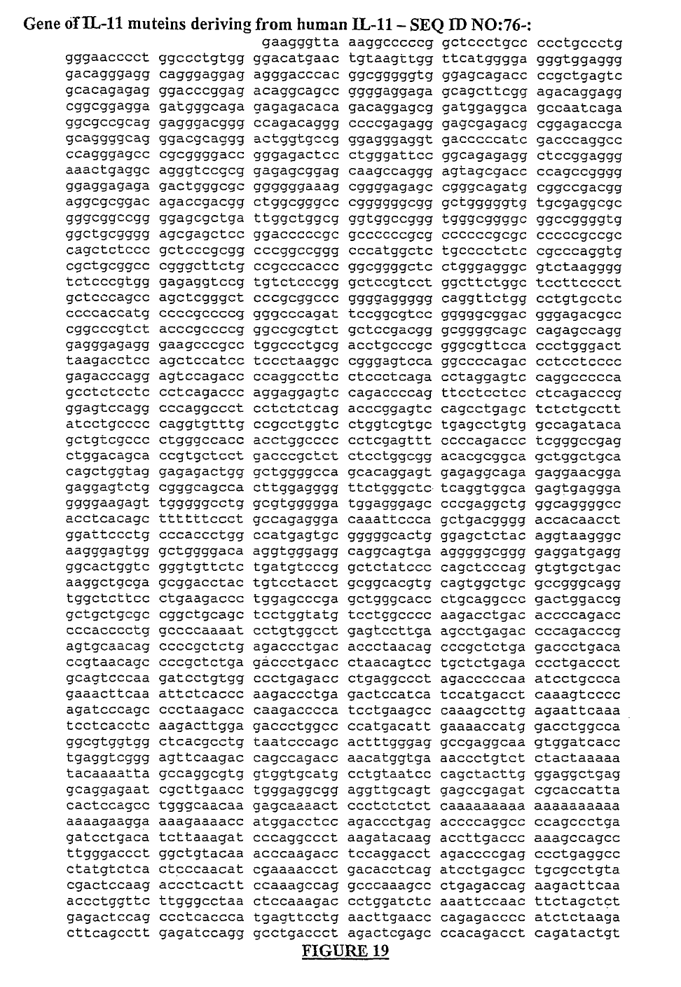

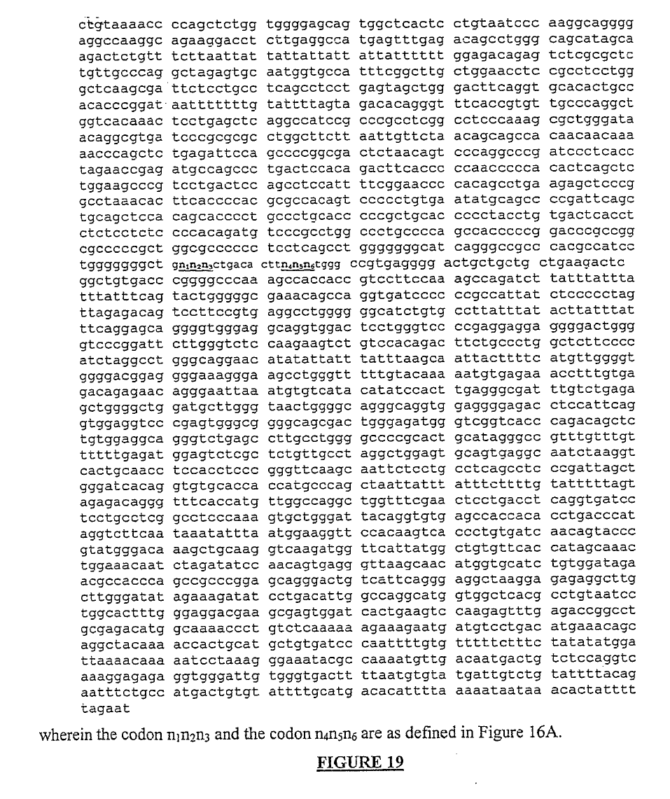

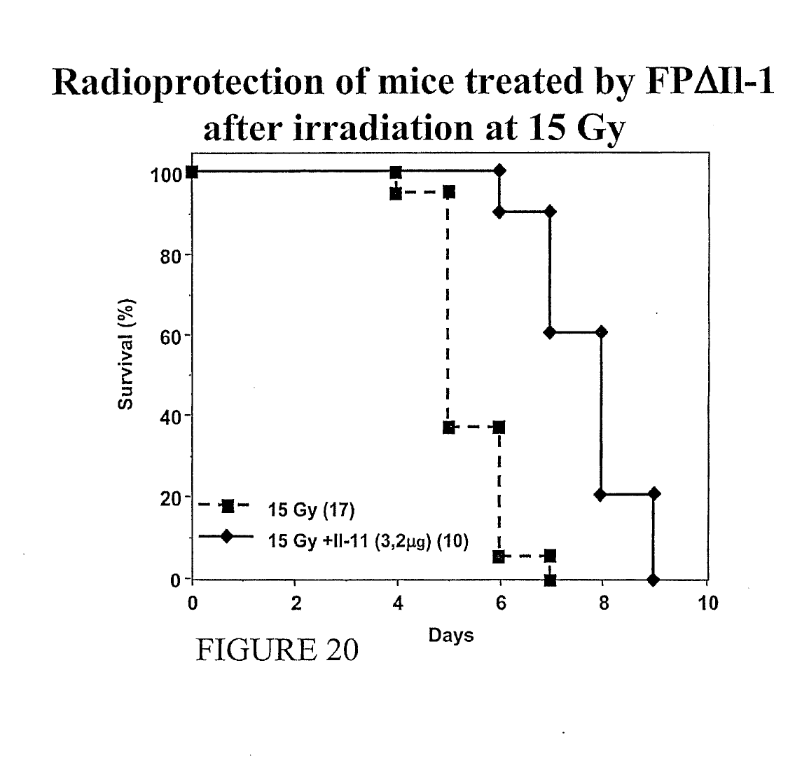

The present invention thus more particularly encompasses any nucleic acid (e.g. DNA) which comprises or consists in the sequence of SEQ ID NO:76 or of SEQ ID NO:74, which are shown on FIGS. 19 and 17, respectively, and wherein the codons n₁n₂n₃ and n₄n₅n₆ are as above-defined.

The sequence of SEQ ID NO:76 corresponds to the human IL-11 wild-type gene (as defined in AY207429 NCBI accession sequence by position 1582 to position 7566), appropriately mutated in accordance with the present invention, i.e. wherein the wild-type codons cac and gac coding for H182 and D186 have been replaced by n₁n₂n₃ and n₄n₅n₆ as above-defined.

The sequence of SEQ ID NO:74 corresponds to the nucleotide sequence of SEQ ID NO:73 (AY207429 NCBI sequence), appropriately mutated in accordance with the present invention, i.e. wherein the wild-type codons cac and gac coding for H182 and D186 have been replaced by n₁n₂n₃ and n₄n₅n₆ as above-defined. Following the same mutational scheme, similarly-mutated sequences can be obtained from wild-type IL-11 non-human DNA, such as macaque, mouse and rat wild-type IL-11 DNA, and such similarly-mutated sequences are encompassed by the present invention.

The present invention also encompasses any nucleic acid comprising or consisting a RNA sequence deriving from a wild-type IL-11 RNA sequence appropriately mutated in accordance with the present invention, i.e. wherein the wild-type codons CAC and GAC coding for H182 and D186 have been replaced by n₁n₂n₃ and n₄n₅n₆ as above-defined.

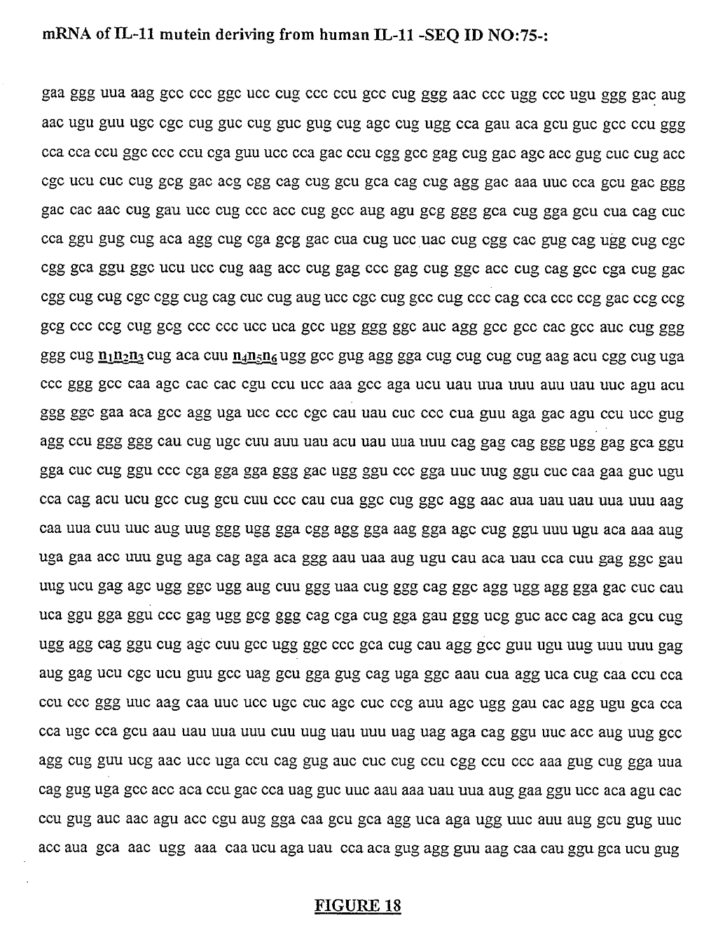

The present invention thus particularly relates to the sequence of SEQ ID NO:75, shown on FIG. 18. The sequence of SEQ ID NO:75 corresponds to the mRNA sequence of human wild-type IL-11 (as defined in AY207429 NCBI accession sequence by joined sequence 1582-1651, 3014-3186, 3386-3472, 3584-3745, 5778-7566), appropriately mutated in accordance with the present invention (n₁n₂n₃ and n₄n₅n₆ are underlined). The present invention also relates to any mutated RNA sequence which is similarly obtained from wild-type non-human IL-11 RNA, such as macaque, mouse, rat IL-11.

According to a further aspect of the present invention, the application also relates to any transfection vector, such as e.g. a plasmid, which comprises a nucleic acid of the present invention, i.e. a nucleic acid coding for an IL-11 mutein of the invention.

Illustrative and useful transfection vectors of the invention comprise those that comprise as an insertion sequence a sequence comprising or consisting of a sequence which derives from a sequence coding for a wild-type IL-11 by replacement of the codons coding for the hydrophilic amino acids in positions 182 and 186 by the codons n₁n₂n₃ and n₄n₅n₆ as above-defined, and possibly by deletion of codons that are not necessary to an biological activity of the IL-11 type, such as e.g. by deletion of the codons which in the complete wild-type IL-11 code for the N-terminal signal peptide and/or the those N-terminal codons corresponding to proline-rich regions.

Illustrative and useful transfection vectors of the invention thus can comprise a sequence comprising or consisting of a sequence which derives from a sequence coding for a wild-type IL-11 by replacement of the codons coding for the hydrophilic amino acids in positions 182 and 186 by the codons n₁n₂n₃ and n₄n₅n₆ as above-defined, and by deletion of those codons of the complete wild-type IL-11 that code for the first 21 N-terminal amino acids or for the first 31 or 34 N-terminal amino acids.

A short nucleotide sequence coding for a Flag tag, such as Asp-Tyr-Lys-Asp-Asp-Asp-Asp-Lys, followed by another short nucleotide sequence coding for a consensus sequence that can be recognized and phosphorylated by a kinase (such as Arg-Arg-Ala-Ser-Val-Ala that can be recognized and phosphorylated on the serine residue by the bovine heart kinase) can be added at one end of the IL-11 mutein encoding nucleic acid, e.g. at the 5′ part of it, in lieu et place of the codons which in the complete wild-type IL-11 code for the first 31 N-terminal amino acids.

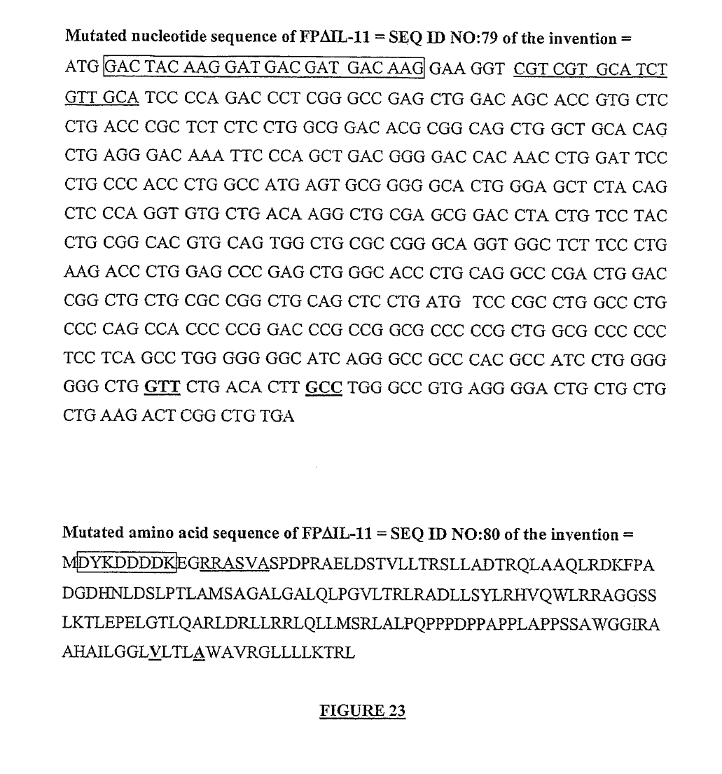

Such a transfection vector is described in example 1 below. An illustrative and useful insertion sequence for such a vector is shown on FIG. 23 under SEQ ID NO:79 (wild-type human IL-11 which has been mutated in accordance with the present invention by the above-defined n₁n₂n₃ and n₄n₅n₆ codons, which has been deleted from the codons coding for the first 31 N-terminal amino acids, and to which codons coding for a Flag tag and for consensus sequence recognized by the heart bovine kinase have been added: in FIG. 23, the Flag tag is boxed, and the consensus sequence for kinase is underlined).

According to a further aspect of the invention, the present application relates to any cell comprising a nucleic acid of the invention, and/or which has been transfected by a tranfection vector of the invention, and/or which express a mutein of the invention. Such cells may e.g. be used to produce and isolate IL-11 muteins of the invention. Any. cell that is available to the person of ordinary skill in the art as appropriate host cell may used to be transformed by a transfection vector of the invention, so that the transformed cell thereby produced can express a mutein of the invention. Appropriate standard host cells e.g. comprise E. coli cells, such as e.g. the E. coli BL21(DE3) strain (available from Novagen).

The invention thus encompasses a method to produce an IL-11 mutein of the invention which comprises culturing a cell of the invention in a suitable culture medium (e.g. for a E. coli transformed cell, in Luria-Bertani medium), and isolating said IL-11 mutein from said cell.

The present invention thus encompasses any cell transformed with a nucleic acid sequence of the invention in operative association with an expression control sequence capable of directing replication and expression of said nucleic acid sequence.

As indicated above and illustrated below, the IL-11 muteins of the invention have at least retained an ability to bind to IL-11Rα and gp130, and to induces an in vitro and in vivo activity of the type induced by wild-type IL-11. They thus are all useful in every application in which wild-type IL-11 is considered as useful.

Exemplary biological or medical applications of IL-11 are described in U.S. Pat. No. 6,126,933; WO 00/74707; U.S. Pat. No. 5,460,810; U.S. Pat. No. 6,540,993; U.S. Pat. No. 5,215,895; WO 00/53214; the contents of which are herein incorporated by reference.

The IL-11 muteins of the invention for which H182 and D186 have been replaced by Val and Ala further prove to be highly more efficient than wild-type IL-11, and may thus be referred to as IL-11 hyperagonists. For example, the H182V+D186A muteins of the invention (i.e. the muteins which comprise a sequence of SEQ ID NO:10, SEQ ID NO:25, SEQ ID NO:40, or SEQ ID NO:55, or conservative variants thereof) bind to IL-11Rα with a three-fold enhanced affinity compared to wild-type IL-11, are 60 to 400 fold more active on the proliferation of 7TD1 murine hydridoma cells than wild-type IL-11, and are required at a ten fold lower dose to induce the same in vivo radioprotection wild-type IL-11 iso-effect (see examples 1 and 2 below).

The present application therefore also encompasses the IL-11 muteins of the invention as agents useful for improving resistance to radiation, such as resistance to radiation therapy for the treatment of cancer or for the preparation of patients for bone marrow transplantation.

The present application also encompasses the IL-11 muteins of the invention as agents useful for improving resistance to deleterious effects induced by chemotherapy for the treatment of cancer.

It more particularly relates to the IL-11 muteins of the invention as anti-thrombocytopenia agents.

The IL-11 muteins of the invention can also be useful as anti-inflammatory agents, and/or as agents to induce or stimulate hematopoiesis, neurogenesis, osteoclastogenesis, and/or female fertility.

Going into details of the anti-inflammatory aspect of the invention, it can be emphasized that, as known by the skilled person, wild-type IL-11 has septic shock-protective and diabetes-protective activities (see introduction). It is also known by the skilled person that the underlying mechanism for these protective IL-11 activities can be found in the inhibition exerted by IL-11 microvascular endothelial apoptosis (see e.g. Sheridan et al. 1999, “Interleukin-11 attenuates pulmonary inflammation and vasamotor dysfunction in endotoxin-induced lung injury”, Am. J. Physiol. 277 (Lung Cell. Mol. Physiol. 21): L861-L867; Nicoletti et al. 1999, “Early prophylaxis with recombinant human interleukin-11 prevents spontaneous diabetes in NOD mice”, Diabetes, vol. 48, pages 2333-2339).

As it is also known that the primary lesion induced by radiation is microvascular endothelial apoptosis (cf e.g. Paris et al. 2001, “Endothelial apoptosis as the primary lesion initiating intestinal radiation damage in mice”, Science vol. 293, pages 293-297), the muteins of the invention are useful agents for inhibiting microvascular endothelial apoptosis. The muteins of the invention hence find advantageous applications in the prevention, and/or symptom alleviation, and/or treatment of those diseases or conditions for which inhibition of microvascular endothelial apoptosis is needed, such as septic shock and diabetes (e.g. type 1 diabetes). The muteins of the invention are powerful anti-inflammatory agents that most probably will be capable of down-regulating early immunodiabetogenic pathways.

The present invention thus relates to any drug that comprises a therapeutically effective amount of an IL-11 mutein of the invention, or a nucleic acid of the invention, or a vector of the invention, or a cell of the invention. Such a drug may further comprise any pharmaceutically-acceptable vehicle (e.g. isotonic sodium chloride solution) that is available to the person of ordinary skill in the art of preparing drugs, as well as any stabilizer, preservative, buffer, antioxidant, or additive that the person of ordinary skill will find appropriate. The drug may be produced in any form and conditioning that is appropriate to its intended mode of administration (parenteral, intravenous, subcutaneous, topical, etc.). The dosage regimen of a drug of the invention will be determined by the attending physician considering the condition, body weight, sex, diet, age, and other medically-relevant features of the patient to be treated. As an advantageous feature of the invention, those drugs which comprise the muteins for which H182 and D186 by Val and Ala will be usually required at lower doses than would have been wild-type IL-11.

A drug of the invention may be useful for stimulating and/or enhancing cells involved in the immune response and cells involved in the proper functioning of the hematopoietic system.

It may also be useful for treating inflammatory bowel diseases (e.g. Crohn's disease, ulcerative colitis, indeterminate colitis and infectious colitis), mucositis (e.g. oral mucositis, gastrointestinal mucositis, nasal mucositis, and proctitis), necrotizing enterocolitis, inflammatory skin disorders (e.g. psoriasis, atopic dermatitis, and contact hypersensitivity), aphtous ulcers, pharyngitis, esophagitis, peptic ulcers, gingivitis, periodontitis, and ocular diseases (e.g. conjunctivitis, retinis, and uveitis).

It may also be useful to prevent or treat hemorrhagic shock, and to protect the gastroinstestinal system during a hemorrhagic shock and resuscitation.

It may also be useful to prevent or treat an immune-mediated cytotoxicity, such as graft versus host disease or rejection of organ and tissue transplants, as well as non-immune-mediated necrotic injuries, such as localized tissue or cell injury caused by loss of blood supply, corrosion, burning, or the local lesion of a disease.

The invention more particularly relates to any anti-thrombocytopenia drug, which comprises a mutein of the invention, and a therapeutically effective amount of an active principle for chemotherapy of cancer.

A drug of the invention is also useful for the prevention, symptom alleviation, or treatment of any disease or condition for which microvascular apoptosis inhibition is desired, and more particularly for the prevention, symptom alleviation, or treatment of of diabetes, such as type 1 diabetes, and/or septic shock.

The following examples are given as illustrative examples, and are in no way intended to restrict the scope of the present invention. More particularly, while human IL-11 muteins are described as a useful and particularly relevant illustration, any conservative variants thereof that the person of ordinary skill in the art will contemplate are encompassed by the present application.

### EXAMPLE 1

### Production of IL-11 Muteins, and Characterization of their Structures, Affinity, Specificity and Cell Line Bioactivity

### EXPERIMENTAL

Bacterial Strains, Enzymes and Chemicals

Escherichia coli DH5α was from Invitrogen Life Technologies. BL21 (DE3) and pET-22b(+) were from Novagen. E. coli recombinant human IL-11 was from PeproTech Inc. (London, UK) and R & D Systems (Wiesbaden-Nordenstadt, Germany). Primers for mutagenesis were from Genset. MAB628 and anti-hIL-11 biotinylated polyclonal antibody BAF218 were from R & D Systems. [γ-³²P]ATP with a specific radioactivity of ˜3000 Ci/mmol was obtained from Amersham Pharmacia Biotech. Acrylamide and N,N′-methylene-bisacrylamide were from Bio-Rad. RPMI-1640, DMEM, glutamine and FCS were from Gibco-BRL. The catalytic subunit of cAMP-indepentent protein kinase from bovine heart muscle, streptavidin conjugated alkaline phosphatase, sodium dodecyl sulfate (SDS) and anti-Flag M2 monoclonal antibody were obtained from Sigma (Bornem, Belgium).

Mutagenesis

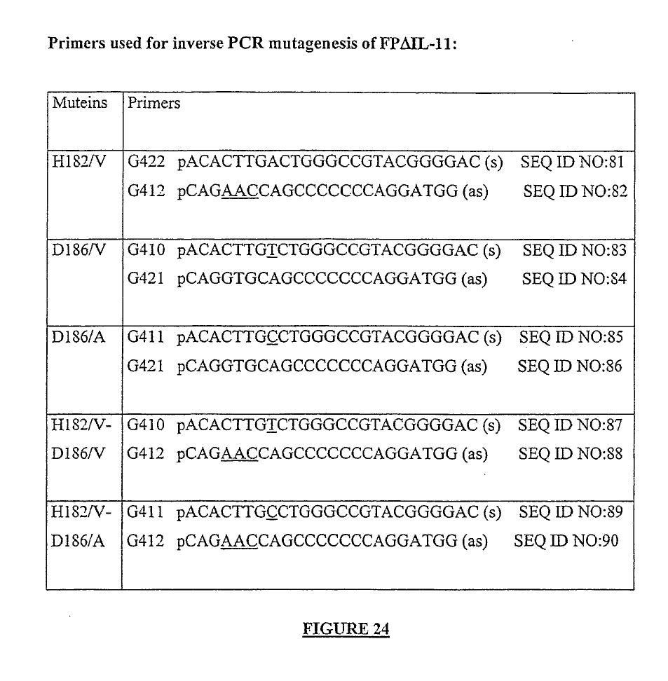

FPΔIL-11 was mutated by inverse PCR amplification of the plasmid pET-FPΔIL-11 previously described in Wang et al. 2002, using the primers shown on FIG. 24, followed by a Dpn I digestion to eliminate the parental plasmid.

For a detailed description of the mutation by inverse PCR amplification, see Stemmer, W. P. and Morris, S. K. (1992) Enzymatic inverse PCR: a restriction site independent, single-fragment method for high-efficiency, site-directed mutagenesis. Biotechniques 13, 214-220, the content of which is herein incorporated by reference.

For the construction of plasmid pET-FPΔIL-11, see Wang, X. M., Wilkin, J. M., Boisteau, O., Harmegnies, D., Blanc, C., Vandenbussche, P., Montero-Julian, F. A., Jacques, Y. and Content, J. (2002) Engineering and use of 32P-abelled human recombinant interleukin-11 for receptor binding studies. Eur. J. Biochem. 269, 61-68, the content of which is herein incorporated by reference.

Please note, in accordance with Wang et al. 2002, the N-terminal nucleotides encoding the first 31 amino acids of human IL-11 joined CDS shown on FIG. 16A under SEQ ID NO:69 have been deleted (the first 21 signal peptide amino acids+the 10 amino acids which follow and which correspond to a proline-rich region), and replaced by a sequence encoding a Flag tag (Asp-Tyr-Lys-Asp-Asp-Asp-Asp-Lys) followed by a consensus amino-acid sequence (Arg-Arg-Ala-Ser-Val-Ala) that can be recognized and phosphorylated on the serine residue by the bovine heart kinase. FPΔIL-11 therefore has the following sequence (upper nucleotide line in italic=human IL-11 joined CDS sequence SEQ ID NO:69, lower nucleotide line=FPΔIL-11; bottom amino acid line=FPΔIL-11 protein):

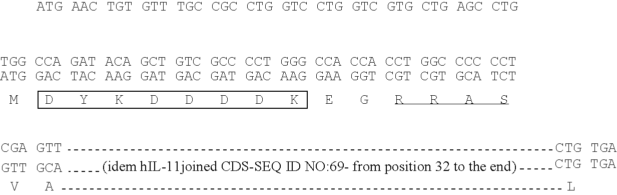

The Flag tag of FPΔIL-11 is boxed; the phosphorylation site recognized by the bovine heart protein kinase catalytic subunit created in FPΔIL-11 is underlined.

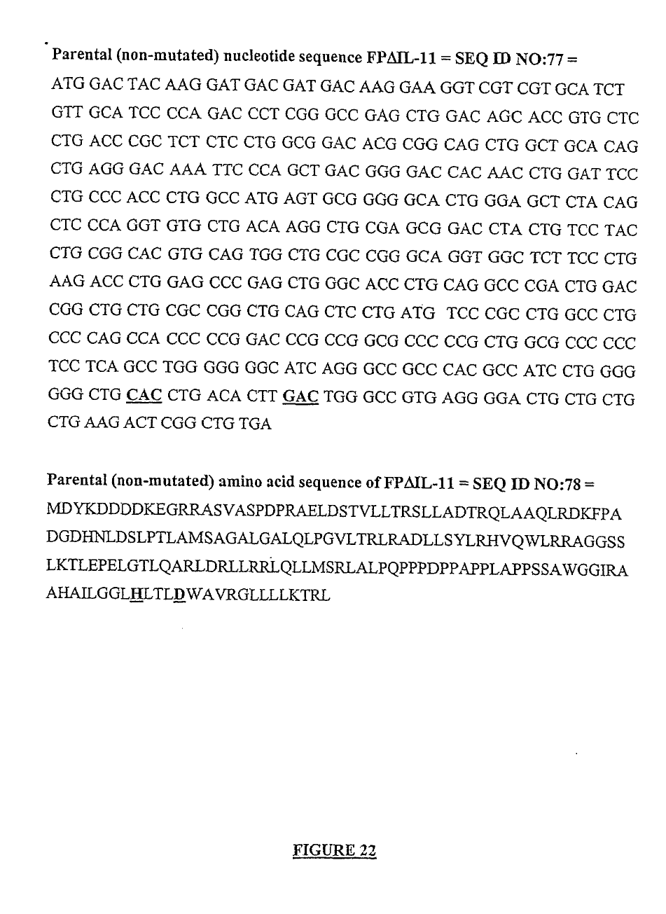

Hence, the nucleotide sequence of the non-mutated parental FPΔIL-11 is the sequence of SEQ ID NO:77, and its amino acid sequence is the sequence of SEQ ID NO:78, shown on FIG. 22.

And, the nucleotide sequence of FPΔIL-11 mutated in accordance with the present invention is the sequence of SEQ ID NO:79, and the amino acid protein sequence of this mutated FPΔIL-11 is the sequence of SEQ ID NO:80, shown on FIG. 23.

Gel purified PCR fragments were ligated overnight at 16° C. using T4 DNA ligase and then used to transform E. coli DH5α. The corresponding plasmids were amplified in DH5α, sequenced and then used to transform the BL21(DE3) strain of E. coli.

Production and Purification of Parental and Mutant FPΔIL-11

BL21 (DE3) cells transformed with the plasmid carrying the mutant or parental FPΔIL-11 cDNA were cultured in Luria-Bertani medium containing 100 μg/ml of ampicillin. Expression of the recombinant proteins was induced by 1 mM IPTG for 2 h at 37° C.

E. coli were then lysed by 30 minutes incubation at 37° C. in presence of 0.1% triton X-100 and 150 μg/ml of lysozyme in 50 mM Hepes, pH 7.4, followed by sonication for 5 minutes at an intensity level of 5 using a microprobe (Vibra Cell, Sonics Materials Inc. Danburg, Connecticut, USA). Lysates were centrifuged two times at 13,000 g for 25 min at 4° C. and then assayed or purified as previously described [Wang et al. 2002, cited supra, and incorporated by reference]. Briefly, lysates were precipitated with 60% (NH₄)₂SO₄ in order to concentrate the crude proteins. Salts were eliminated by dialysis against 50 mM Hepes, pH 7.4 buffer before the purification of samples by chromatography on a Mono-S HR5/5 column (Amersham Pharmacia Biotech) using a 50 mM Hepes buffer, pH 7.4, and a 0-1 M NaCl gradient.

Quantification of Parental and Mutant FPΔIL-11 by ELISA

Two antibodies raised against human IL-11, a non-neutralising monoclonal antibody MAB618 and a biotinylated polyclonal BAF218, were used to quantify the recombinant human parental and muteins by sandwich ELISA method. 96-wells plates were coated overnight at 4° C. with 100 μl of monoclonal antibody MAB618 at a concentration of 2 μg/ml. After blocking with 3% BSA, 100 μl of serial dilution of samples were added and incubated for 1 hour at 37° C. After washing with PBST buffer (PBS buffer in the presence of 0.1% of Tween 20), plates incubated for another hour at 37° C. with 100 μl/well of biotinylated polyclonal antibody BAF218 at a concentration of 30 ng/ml. Before another incubation at 37° C. for 1 hour with straptavidin conjugated alkaline phosphatase (1/5000), the plates were washed 3 times with TBS buffer (100 mM Tris-HCl, 150 mM NaCl, pH 7.5). Finally, the test was revealed using an ELISA Amplification System (Gibco BRL). Commercial recombinant IL-11 was used as a standard and the sensitivity was 2 pg/ml.

Mass Spectrometry

The exact molecular weight of the FPΔIL-11 and the mutein was determined using nano-electrospray mass spectrometry on a hybrid quadrupole Time-of-Flight Q-TOF mass spectrometer (Micromass, Whytenshawe, UK). Prior to analysis, samples were desalted using Vivaspin microconcentration devices with a cut-off of 10 kDa (Millipore, Bedford, Mass.). After washing twice with water, samples were dissolved in a mixture of 50% acetonitrile and 0.1% formic acid in water to a concentration of approximately 5 pmol/μl. Four μl of this sample were loaded in a nano-electrospray capillary (MDS Proteomics, Odense, Dk) that was then placed in the special holder delivered with the instrument. Spray was initiated by slightly breaking the needle tip and supplementing a small back-pressure of nitrogen. The capillary voltage was set at 1250 V. Spectra were accumulated for about 5 minutes, collecting data from m/Z 1000 to 2500 at 1 sec per scan. Data processing was performed using the Masslynx and MaxEnt software delivered with the instrument.

Infrared Spectrometry

ATR-FTIR spectra were recorded at room temperature on a Bruker IFS55 FTIR spectrophotometer equipped with a liquid nitrogen-cooled mercury-cadmium-telluride (MCT) detector at a nominal resolution of 2 cm⁻¹ and encoded every 1 cm⁻¹. The internal reflection element (IRE) was a germanium plate (50×20×2 mm) with an aperture angle of 45°, yielding 25 internal reflections. The spectrophotometer was continuously purged with air dried on a FTIR purge gas generator 75-62 Balston (Maidstone, England) at a flow rate of 10-20 l/min in the sample compartment and 5 l/min in the optic compartment. Thin films were obtained by slowly evaporating a sample under a stream of nitrogen on one side of the ATR plate [Fringeli and Gunthard (1981). Infrared membrane spectroscopy. Mol. Biol. Biochem. Biophys. 31, 270-332, the content of which is herein incorporated by reference]. The ATR plate was then sealed in a liquid sample holder. The sample on the ATR plate was rehydrated by flushing ²H₂O-satured N₂, at room temperature. 256 scans were averaged for each measurement. Secondary structure determination was based on the shape of the amide I band (1600-1700 cm⁻¹), which is sensitive to the secondary structure [Goormaghtigh et al. (1990). Secondary structure and dosage of soluble and membrane proteins by attenuated total reflection Fourier-transform infrared spectroscopy on hydrated films. Eur. J. Biochem. 193, 409-420, the content of which is herein incorporated by reference].

Hydrogen/deuterium exchange kinetics: nitrogen was saturated with ²H₂O by bubbling in a series of three vials containing ²H₂O. Before starting the deuteration, 10 spectra of the sample were recorded to test the stability of the measurements. At zero time, the ²H₂O-saturated N₂ flux, at a flow rate of 100 ml/min (controlled by a Brooks flow meter), was connected to the sample. For each kinetic time point, 24 scans were recorded and averaged at a resolution of 4 cm⁻¹. All the spectra of the kinetics were corrected for atmospheric water absorption and side chain contribution. The subtraction of atmospheric water was done automatically by a home-written software which computed the subtraction coefficient as the ratio of the atmospheric water band between 1579 and 1572 cm⁻¹ on the sample spectrum and on a reference atmospheric water spectrum [4549]. The area of amide II, characteristic of the δ(N—H) vibration, was obtained by integration between 1596 and 1502 cm⁻¹. For each spectrum, the area of amide II was divided by the corresponding amide I ν(C=O) area. This ratio expressed in percentage was plotted versus deuteration time. The 100% value is defined by the amide II/amide I ratio obtained before deuteration. The 0% value corresponds to a zero absorption in the amide 11 region, observed for a full deuteration of the protein.

Circular dichroism

CD measurements were carried out on a Jasco J-720 spectropolarimeter (Japan Spectroscopic Co., Ltd., Tokyo, Japan) equipped with a temperature control unit and calibrated according to Chen and Yang [Chen and Yang (1977). Two-point calibration of circular dichrometer with d-10-camphorsulfonic acid. Anal. Lett. 10, 1195-1207, the content of which is herein incorporated by reference]. The spectral bandwidth was 2 nm (<250 nm) and 1 nm (>250 nm), respectively. The measurements were carried out at a temperature of 20° C., the solvent was PBS throughout. The time constant ranged between 1 and 4 s and the cell path length between 0.1 and 10 mm.

Labelling of FPΔIL-11 and of its Mutein

FPΔIL-11 and H/V-D/A were labelled through protein phosphorylation with [γ-³²P]ATP in the presence of bovine heart kinase and phosphorylation was checked by autoradiography as previously described [Wang et al. (2002), cited supra, and incorporated by reference].

SDS-PAGE and Western Blot

SDS-PAGE was carried out as previously described [Laemmli (1970). Cleavage of structural proteins during the assembly of the head of bacteriophage T4. Nature 227, 680-685, the content of which is herein incorporated by reference]. Muteins and parental FPΔIL-11 were transfered from gels to a nitrocellulose membrane and detected by incubation with biotinylated goat polyclonal antibody BAF218 (R&D), then streptavidin-conjugated alkaline phosphatase and finally revealed with the NBT/BCIP system (Sigma). Alternatively, proteins were detected using a biotinylated anti-flag antibody (M2 antibody, from Sigma).

Binding of ³²P-H/V-D/A to Cells

Binding of ³²P-H/V-D/A on 7TD1 cells was carried out as described for the parental ³²P-FPΔIL-11 on B13Rα1 cells by Wang et al [Wang et al. (2002), cited supra, and incorporated by reference]. 7TD1 cells (5×105) were pre-incubated in culture medium lacking growth factor for 2 h and were washed 3 times with phosphate-buffered saline, pH 7.4 (PBS). For binding studies, radiolabelled H/V-D/A was added to cells at the indicated concentration in PBS containing 0.5% bovine serum albumin. The mixture was incubated at 4° C. for the appropriate time and bound radiolabelled H/V-D/A was separated from the free radioactivity by centrifugation at 3000 g for 1 min through a 0.2 ml layer of a mixture of 40% dioctyl phthalate and 60% dibutyl phthalate (Janssen Chimica, Beerse, Belgium). After quick freezing, the tip of each tube containing the cell pellet was cut-off and radioactivity was counted in a Beckman β-counter. Non-specific binding was determined by incubating cells with radiolabelled H/V-D/A in the presence of a 200-fold molar excess of unlabelled H/V-D/A.

Surface Plasmon Resonance Studies

These experiments were performed with a BiaCore 2000 optical biosensor (BiaCore, Uppsala, Sweden). A fusion protein of human IL-11R and IL-2 (IL-11R-IL-2) [Blanc et al. (2000). Monoclonal antibodies against the human interleukin-11 receptor alpha-chain (IL-11Ralpha) and their use in studies of human mononuclear cells. J. Immunol. Methods 241, 43-59, the content of which is herein incorporated by reference] was coupled through primary amino groups to a carboxymethyl dextran flow cell (CM5) at a low immobilisation level (about 500 RU per flow cell) compatible with kinetic binding studies. Subsequent binding of parental FPΔIL-11 or mutein was carried out in Hepes-buffered saline (pH 7.4) at a flow rate of 10 μl/minute at room temperature.

IL-11 Bioassay

IL-11 activity was measured using the 7TD1 cells. 2×10³ cells/well were cultured in flat-bottom 96 wells microtiter plates during 7 days in the presence of serial dilutions of the purified mutein or parental FPΔIL-11, or E. coli crude lysates containing different muteins previously adjusted to the same protein concentration. The cell number in each well was then determined by a colorimetric assay for hexosaminidase [Van Snick et al. (1986). Purification and NH2-terminal amino acid sequence of a T-cell-derived lymphokine with growth factor activity for B-cells hybridomas. Proc. Natl. Acad. Sci. U.S.A. 83, 9679-9683, the content of which is herein incorporated by reference]. Bioactivity was assayed similarly on 1×10⁴ B9 cells/well for about 3 days and revealed by XTT calorimetric assay. Each sample was tested in triplicate using a commercial recombinant human IL-11 (from PeproTech) as a standard.

Results

Expression, Purification and Initial Characterisation of the H/V-D/A Mutein

FPΔIL-11 was used as the human IL-11 parental molecule to generate IL-11 muteins by mutagenesis because i) it has the same biological activity as the wild-type human recombinant IL-11 and ii) the presence of the flag-tag (F), the phosphorylation site (P) and the absence of the first ten amino acids of IL-11 (Δ) allow a strong expression, a simple purification and an easy radio-labelling of IL-11 [Wang et al. (2002), cited supra].

To evaluate the involvement of H182 and D186 residues with respect to biological activity and receptor binding, the corresponding positions were substituted by site-directed mutagenesis using an inverse PCR method [Stemmer and Morris (1992), cited supra, and incorporated by reference]. These two residues were replaced by a valine (H182V) and an alanine (D186/A) to generate a mutein named H/V-D/A.

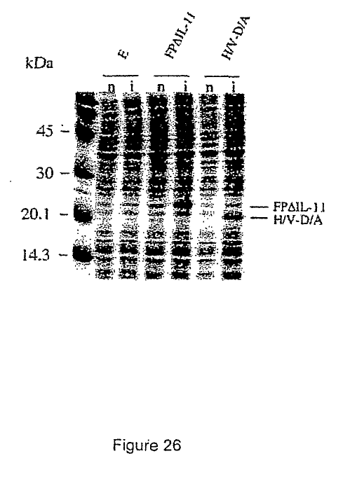

The expression of these parental and mutant FPΔIL-11 in E. coli was analysed by SDS-PAGE (FIG. 26). The parental molecule had an apparent molecular mass of about 24 kDa, a value higher than its theoretically expected one (20.050 kDa). This difference could be due to the introduction of numerous charged residues present in the flag-tag and the phosphorylation site at the N-terminus of FPΔIL-11 (1 Glu, 5 Asp, 2 Arg and 2 Lys). Indeed, when the two charged residues H182 and D186 of FPΔIL-11 were replaced by two hydrophobic amino acids, the resulting mutein moved faster in gels than its parent, so that its apparent molecular mass (19 kDa) was close to its calculated one (19.9 kDa). This observation reinforced the hypothesis that the charged residues could influence the molecular mobility in SDS-PAGE. However, to rule out the possibility that the reduced mobility of the H/V-D/A could be linked to a truncation of the protein, purified parental and mutant FPΔIL-11 were submitted to mass spectrometric analysis. FPΔIL-11 and H/V-D/A were found to have masses of 20.016 kDa and 19.934 kDa respectively, in perfect agreement with their predicted molecular masses.

Even though the increased electrophoretic mobility of the H/V-D/A mutein on SDS-PAGE is most likely due to charge modifications, we can not rule out the possibility that it would be partially due to a structural and/or conformational change of the molecule induced by mutagenesis. Such changes could render the mutein more compact than the parental molecule, therefore making it more resistant to heat denaturation and move faster in polyacrylamide gels.

Structural Analysis by Infrared Spectrometry (IR) and Circular Dichroism (CD)

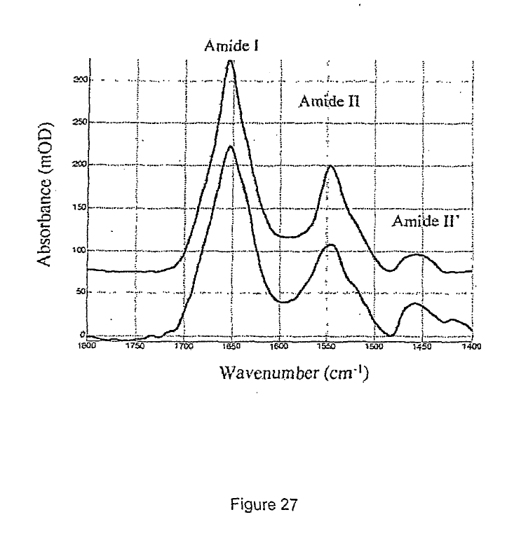

In order to further evaluate a potential conformational change induced by mutagenesis, the parent and mutant proteins purified to homogeneity were characterized by attenuated total reflection Fourier transform infrared spectrometry (ATR-FTIR). This technique has been successfully used to investigate the structure of soluble and membrane proteins [Goormaghtigh et al. (1990), cited supra]. The method is based on the analysis of the vibration bands of protein and particularly the amide I band, ν(C=O), whose absorption frequency is dependent upon the secondary structure. FIG. 27 represents the ATR-FTIR deuterated spectra of those two proteins recorded at pH 7.4. Their similar spectra suggest that the replacement of two amino acids (H182 and D186) by a valine and an alanine, respectively, does not have a detectable effect upon the protein secondary structure. The main absorption peak within the amide I is located in a region associated to the α-helical structure, confirming that this structure is predominant in both IL-11 (parent and mutein).

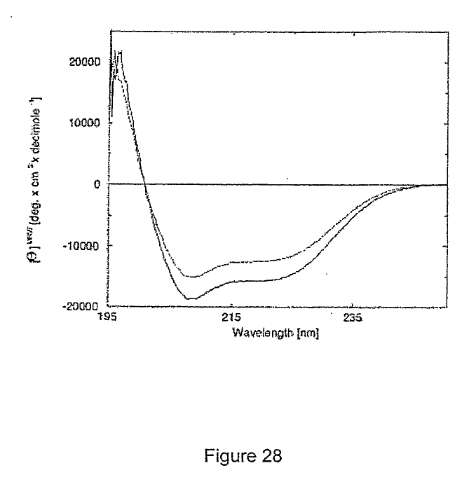

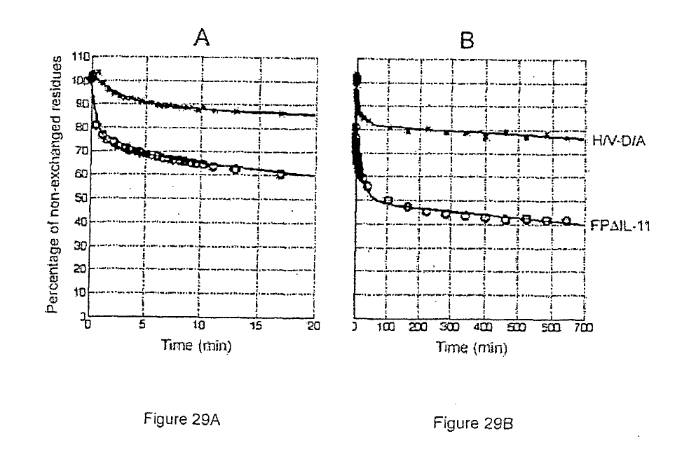

Parental and mutant IL-11 were also submitted to CD analysis because this technique is more sensitive to cc-helical structures. FIG. 28 shows their CD spectra. Both spectra have the same shape but their intensity is different. Secondary structure analysis [Kalai et al. (1997). Analysis of the human interleukin-6/human interleukin-6 receptor binding interface at the amino acid level: proposed mechanism of action. Blood 89, 1319-1333, the content of which is herein incorporated by reference] of the far UV CD spectrum of both proteins reflect the α-helical character of the proteins parental IL-11: α-helix 44.8%, β-sheet 14.0%, turn 15%, remainder 26.2%; mutant IL-11: α-helix 38.8%, β-sheet 17.0%, turn 15.7%, remainder 28.5%), which are typical for a four-helix bundle cytokine. The somewhat lower helical content of the IL-11 mutein compared to the parental might reflect a conformational change introduced by the mutated amino acids.

To further characterize conformational changes taking place upon mutagenesis of FPΔIL-11, deuteration kinetics of the mutein and its parental protein were measured. In a soluble protein, the rate of hydrogen/deuterium exchange is essentially related to protein structure stability (local unfolding dynamics in secondary structures govern the exchange). The hydrogen exchange rate of the proteins was followed by monitoring the amide II absorbance peak decrease [67 (N—H) maximum in the 1596-1502 cm⁻¹ region] because of its shift to the 1460 cm⁻¹ region [amide II′, δ(N-D)] upon deuteration (data not shown). The variations with time of the percentages of non-exchanged residues, calculated from the ratio of amide II/amide I as described in Experimental, are shown in FIG. 29. It appears that the FPΔIL-11 is undergoing a fast exchange, whereas H/V-D/A mutein is more resistant to hydrogen/deuterium exchange, suggesting that the mutein might form oligomers and/or have a more compact structure than parental FPΔIL-11.

By gel-filtration on a Superdex-75 column, parental and mutant proteins were both eluted at a similar position corresponding to a monomeric form (FIG. 30), indicating that the increased hydrophobicity due to mutagenesis at site I did not lead to the formation of dimers or oligomers.

Interaction with Soluble IL-11Rα

In order to find out if mutagenesis and associated conformational change of H/V-D/A have an effect on its interaction with IL-11Rα, the association and dissociation kinetic constants (kₒₙ, k_{off}) describing parent IL-11 and H/V-D/A mutein binding to human IL-11Rα were determined by surface plasmon resonance biosensor analysis using dextran-immobilized purified human IL-11Rα-IL-2 [Blanc et al. (2000), cited supra] fusion protein as matrix. As depicted in Table 1 below, the association (kₒₙ) and dissociation (k_{off}) kinetic constants of H/V-D/A were both much higher (35 and 14 fold respectively) than those of parental FPΔIL-11, leading to an equilibrium dissociation constant (K_{d}) for the mutein that was 3-fold lower than for FPΔIL-11.

TABLE 1
Kinetic (kₒₙ association, k_{off} dissociation) and equilibrium
(K_{d} dissociation) constants for the binding of FPΔIL-11
and H/V-D/A to the recombinant human IL-11R-IL-2, determined
by surface plasmon resonance.

| IL-11 | kₒₙ (M⁻¹s⁻¹) | k_{off} (s⁻¹) | K_{d} (nM) |
|---|---|---|---|
| FPΔIL-11 | 5.90 (±0.90) × 10³ | 9.75 (±0.05) × 10⁻⁴ | 165 (±25) |
| H/V-D/A | 2.30 (±0.74) × 10³ | 1.34 (±0.46) × 10⁻² | 58 (±1.5) |

If one translates the equilibrium dissociation constants in terms of free energies of interaction (AG=−RTln(1/K_{D})), binding of FPΔIL-11 or H/V-D/A to IL-11R-IL-2 is accompanied by free energy changes of 9.2 or 9.8 kcal/mol, respectively, indicating that the mutagenesis and its induced conformational change favour IL-11 interaction with the IL-11Rα receptor.

Interaction with Cell Surface IL-11 Receptors

B13Rα1 and 7TD1 cells were used to test H/V-D/A binding to human and murine IL-11 receptors. B13Rα1 are Ba/F3 cells stably transfected with human gp130 and hIL-11Rα [Lebeau et al. (1997). Reconstitution of two isoforms of the human interleukin-11 receptor and comparaison of their functional properties. FEBS Lett. 407, 141-147, the content of which is herein incorporated by reference]. Non-specific binding component, determined by adding a 200-fold molar excess of unlabelled H/V-D/A, was low (less than 5% of the total association). Analysis of the specific binding data by the method of Scatchard indicated the existence of a single class of binding sites (see Table 2 below).

TABLE 2
Dissociation constants and numbers of sites per cell of FPΔIL-11
and H/V-D/A binding on B13Rα1 and 7TD1 cells

| | | | 7TD1 | | | |
|---|---|---|---|---|---|---|
| | B13Rα1 | | Class 1 sites | | Class 2 sites | |
| | K_{d} | Sites/ | K_{d} | Sites/ | K_{d} | Sites/ |
| Ligands | (nM) | cell | (nM) | cell | (nM) | cell |
| ³²P-FPΔIL-11 | | | | | | |
| competed with FPΔIL-11 | 0.44 | 3079 | 7.20 | 391 | 0.65 | 16 |
| competed with H/V-D/A | 0.40 | 2900 | ND* | ND | ND | ND |
| ³²P-H/V-D/A | | | | | | |
| competed with H/V-D/A | 0.71 | 3462 | 2.70 | 486 | 0.60 | 16 |
| competed with FPΔIL-11 | 0.72 | 3531 | ND | ND | ND | ND |

*ND: non determined

We could only detect high affinity receptors on these cells probably because of an excess of gp130 expression on the surface of the transfected cells. The dissociation constant for the mutein (K_{d}=0.7 nM) was higher than that for its parent (K_{d}=0.4 nM). Binding of ³²P-H/V-D/A could be completely inhibited by an excess of FPΔIL-11 and the reverse was also found, showing that the two molecules compete with each other for this binding.

7TD1 is a murine hybridoma cell line resulting from the fusion of the mouse myeloma cell line Sp2/0-Ag14 with spleen cells from a C57BL/6 mouse. This cell line is well known to respond to picogram amounts of IL-6 [Van Snick et al. (1986), cited supra], but has also a proliferating response to nanogram amounts of IL-11 [Wang et al. (2002), cited supra].

When 7TD1 cells were used for ³²P-labelled H/V-D/A or FPΔIL-11 receptor binding assays, two classes of binding sites were observed (see the above Table 2): low affinity receptors with K_{d} in the nanomolar range likely corresponding to the binding of IL-11 or mutein to isolated IL-11Rα chains, and high affinity receptors with K_{d} in the picomolar range likely corresponding to the association of IL-11/IL-11Rα with gp130 transducing subunits. Similar numbers of either types of receptors were detected with labelled FPΔIL-11 and H/V-D/A, in agreement with the above observation that the two molecules compete for common receptors. In the context of low affinity binding to isolated IL-11Rα chains, the affinity of H/V-D/A (K_{d}=2.7 nM) was found to be around 3-fold higher than that determined for FPΔIL-11 (K_{d}=7.2 nM), in agreement with the biosensor experiments (see the above Table 1). In the context of the high affinity receptor complex however, no differences were found between H/V-D/A and FPΔIL-11 binding (K_{d}=0.60 nM vs 0.65 nM).

Induction of Cell Proliferation

To investigate to what extent the increased affinity of the mutein for the IL-11Rα could impact on its bioactivity, cell proliferation assays were conducted on different cell lines.

As shown in FIG. 31A, H/V-D/A mutein, like IL-11, supports 7TD1 cell proliferation dose-dependently. However, the concentration of the mutein required to induce half-maximal proliferation (EC₅₀) was much lower (400-fold) than that required for the wild-type IL-11 (EC₅₀=0.03 ng/ml for H/V-D/A vs 15 ng/ml for FPΔIL-11 and rhIL-11). This increased activity of the mutein was consistently found in several experiments, with a H/V-D/A/FPΔIL-11 activity ratio ranging from 60 to 400. Gel filtration experiments (FIGS. 30A and 30B) showed that parental and mutant IL-11 behaved as monomeric molecules (at about 20 kDa) with no sign of aggregation, and biological activity was fully associated with these monomers.

In sharp contrast to what was found on 7TD1 cells, the H/V-D/A mutein was about 10-fold less active on B9 cells (FIG. 31B), another murine hybridoma cell line, indicating that the mode of action of the IL-11 mutein was more complex than expected.

In order to check if the H/V-D/A activity was mediated through gp130 transduction, we used an anti-IL-11 mAb (H2) that has been demonstrated to react with an epitope localized in site II of IL-11 [Blanc et al. (2000), cited szipra]. By interfering with gp130 recruitment, this antibody inhibits the binding of FPΔIL-11 to its receptors and consequently inhibits IL-11-dependent cell proliferation [Wang et al. (2002), cited supra]. FIG. 32 shows that this neutralizing antibody is able to inhibit 7TD1 cell proliferation induced by both the parental and mutant FPΔIL-11, indicating that the epitope recognized by the antibody H2 (site 11) is conserved on H/V-D/A mutein, and that H/V-D/A, like parental IL-11, requires the gp130 subunit for exerting its bio-activity. The anti-human gp130 antibodies MAB628 and B-R3 did not affect parental or mutant IL-11 proliferation of the murine 7TD1 cells, and served as controls. As far as these two antibodies have been shown to inhibit cell proliferation on human cells [Chevalier et al. (1996). Interleukin-6 family of cytokines induced activation of different functional sites expressed by gp130 transducing protein. J. Biol.Chem. 271, 14764-14772], these results also indicate that the epitopes recognized by these antibodies on human gp130 are not shared by murine gp130.

When analysing the dose-response curves depicting the inhibitory effect of H2 antibody (FIG. 32), it appeared that the concentration of H2 necessary to induce half-maximal inhibition (IC₅₀) was about 10 fold lower in the case of the H/V-D/A mutein than in the case of parental IL-11. This indicates that the H/V-D/A mutations at site I induce a conformational change at site II that results in an increased affinity for the H2 antibody. Other experiments showed that H/V-D/A, like IL-11, was able to stimulate the proliferation of Ba/F3 cells co-transfected with human IL-11Rα and human gp130, whereas Ba/F3 cells only transfected with human gp130 were insensitive to either molecule. Therefore H/V-D/A, like parental IL-11, cannot activate gp130 in the absence of IL-11Rα.

Relative Roles of H182 and D186 in the Properties of H/V-D/A

In order to investigate the relative importance of H182 and D186, these residues were mutated separately or in combination generating H/V, D/N, D/A and H/V-D/V muteins, in addition to H/N-D/A. As shown in FIG. 33, SDS-PAGE and Western blot analysis indicate a good expression of all recombinant proteins. As observed before for H/V-D/A, all muteins showed systematic differences between their apparent molecular mass on SDS-PAGE and their predicted one. Muteins D/V and D/A moved faster than the mutein H/V, suggesting that the negatively charged residue (D) had more impact on the molecular mobility in gels than the positively charged one (H). The difference of mobility between D/V and D/A also indicated that the charge is not the only factor involved in the mobility change. This reinforces our previous hypothesis that beyond the charge, an SDS resistant conformational change of the molecules resulting from the mutagenesis could also contribute to the mobility change. The two double muteins H/V-D/V and H/V-D/A had similar and higher mobilities than the single muteins, indicating cumulative effects of the two mutations.

7TD1 cells were used to measure the bioactivity of the various FPΔIL-11 muteins (FIG. 34). It appeared that the D/A mutation alone resulted in a strong increase in activity, even stronger than the H/V-D/A combination. The D/V mutation also resulted in an increase in activity but to a far lower extent than the D/A mutation. In contrast, the H/V mutation always resulted in a slight reduction of bioactivity: H/V, H/V-D/V and H/V-D/A were less active than wild-type, D/V and D/A respectively.

These results suggest that D186 is a key amino acid in site I and plays an essential role in the activity of IL-11. Of note, replacement of D186 by a valine instead of an alanine resulted in a much lower increase of activity, suggesting that in addition to the hydrophobic nature, the size of the side chain at position 186 is crucial for this enhancement of activity. The H182 residue also appears to be involved in the interaction at site I but with a minor role.

Discussion

The aim of this study was to create potent agonists of human IL-11 by changing amino acids located in the area (site I) responsible for binding to the specific receptor chain (IL-11Rα). A model of IL-11 (FIG. 25) was built by homology considerations based on the known receptor interaction sites of the related cytokines IL-6, CNTF, and LIF [Jacques et al. (1998). The interleukin-11/receptor complex: rational design of agonists/antagonists and immunoassyas potentially useful in human therapy. Res. Immunol. 149, 737-740, the content of which is herein incorporated by reference]. Supported by mutagenesis experiments, the model predicts that the main energy for receptor ligand binding is provided by hydrophobic interactions of a few apolar side chains shielded by a surrounding scaffold of polar or charged residues which guarantee the specificity of the interaction by the formation of hydrogen bonds and salt bridges [Kalai et al. (1997), cited supra]. Therefore, in order to enhance the interaction of IL-11 with its α-receptor subunit, we replaced two charged amino acids residues H182 and D186 located in the middle of the site I hydrophobic cluster by two hydrophobic ones. We anticipated that increasing locally the hydrophobicity on the surface of site I could influence the quaternary structure of the molecule: a putative large hydrophobic interaction zone generated by mutagenesis might favour H/V-D/A to form oligomers. Superdex-75 chromatography has evidenced that H/V-D/A is in fact expressed as a soluble functional monomeric protein. However, IR hydrogen/deuterium exchange kinetics showed that the H/V-D/A mutein is more resistant to ¹H/²H exchange, suggesting that the mutein might have a more compact structure than parental FPΔIL-11. IR ¹H/²H kinetic studies were indeed recorded at a higher protein concentration since the proteins were concentrated in a film for that experiment. It is then conceivable that additional interactions are present in the IR experiment. Yet, such local interactions encompassing the new, more hydrophobic, domain found in the H/V-D/A mutant could not explain the large effect reported on FIG. 29 where almost 40% of the residues experience a slower exchange, nor can such a difference be explained in view of the minor differences in the secondary structures. On the other hand, a more compact structure is deduced from the mutant's faster mobility on SDS-PAGE, in good agreement with the slower IR ¹H/²H exchange and CD data.

Analysis of the binding characteristics of the H/V-D/A mutein confirmed that residues at the end of the D-helix are implicated in recognition for and interaction with IL-11Rα. Indeed, biosensor studies showed that the H/N-D/A mutations were associated with modifications in the parameters of binding to the isolated IL-11Rα chain. Both the association and dissociation constants were markedly increased, indicating that the nature of the molecular bonds involved in the cytokine-receptor interaction at site I were strongly modified. Despite these changes, the binding affinity of the mutein for IL-11Rα was only three-fold higher than that of parental IL-11. Equilibrium studies on cell surface receptors confirmed this three-fold increase in affinity and further showed that in the context of the high affinity IL-11Rα/gp130 complex, the mutein and wild type IL-11 displayed similar affinities.

The relative bioactivity of the H/V-D/A mutein as compared to wild type IL-11 was not correlated to the difference in affinity between the two molecules. Indeed, on the 7TD1 murine hybridoma cell line, the H/V-D/A had a considerably (up to 400-fold) increased activity, whereas on another murine hybridoma cell line (B9), its bioactivity was reduced by about 10-fold. Such variations are in line with a previous study showing that, on another murine plasmocytoma cell line (T10), the substitution of the D186 by an alanine (D/A mutein) rendered the cytokine 500-fold less active than the wild-type [Czupryn et al. (1995), Ann. New York Acad. Sci. 762, 152-164, cited supra and herein incorporated by reference].

What makes the H/V-D/A more active on 7TD1 cells? Since 7TD1 cells are highly responding to IL-6, such a high H/V-D/A bioactivity could result from their stimulation via IL-6Rα-mediated signal transduction. As parental FPΔIL-11 was found in this study to fully compete with ³²P-labelled H/V-D/A for its high affinity binding to 7TD1 cells and since the binding of this radio-labelled protein to IL-6Rα was not detectable in a RIA assay, this hypothesis can be refuted. The induction by H/V-D/A of murine IL-6 can be also excluded since we found that H/V-D/A bioactivity on 7TD1 was not modified in the presence of an IL-6 neutralising antibody. One has therefore to hypothesize that another factor, whose expression is cell line dependent, is responsible for the enhanced activity of the H/V-D/A mutein. Such a factor could be another unknown receptor chain participating to the structure of the functional IL-11 receptors. The stoichiometry of IL-11 ligand-receptor complex is still an open question and a transducing subunit different from gp130 might participate in IL-11 mediated signal transduction. A possible candidate for this unknown subunit is the gp130-like receptor (GLM-R) that has been recently identified and found to be expressed predominantly on activated monocytes [Ghilardi et al. (2002). A novel type I cytokine receptor is expressed on monocytes, signals proliferation, and activates STAT-3 and STAT-5. J. Biol. Chem. 277, 16831-16836, the content of which is herein incorporated by reference]. This receptor is able to transduce a proliferation signal and induce activation of the transcription factors STAT-3 and STAT-5. Even though its ligand has not yet been identified, GLM-R was not found to be <<per se>> a receptor for IL-11.

In the frame of such a hypothesis (heterocomplex of gp130 with gp130-like receptor), one could postulate that the conformational change induced by mutagenesis could render the mutein H/V-D/A more prone than wild type IL-11 to recruit and/or activate this unknown gp130-like factor. As far as our studies on 7TD1 cells showed that H/V-D/A and wild type IL-11 displayed similar high affinity binding, the higher activity of H/V-D/A would be related to a higher signal transduction efficiency. Therefore, on cells that would express gp130-like in excess to gp130 (like 7TD1), the mutein would be more active, and conversely on cells (like B9 or T10) that would express gp130 in excess to gp130-like the mutein would be less active. Neutralizing antibodies inhibition experiments showed that site II of H/V-D/A remained functional, although its conformation was modified with respect to antibody H2 binding. Such a modification at site II could lead to the recruitment by H/V-D/A of a gp130-like molecule instead of gp130. Alternatively, site II of H/V-D/A would still be involved in recruitment of gp130 and site III involved in recruitment of gp130-like.

In conclusion, we have generated novel hIL-11 muteins with enhanced affinity for IL-11Rα and strongly enhanced activity on 7TD1 cells. These muteins therefore constitute agonist molecules potentially useful in pathologies in which IL-11 has been shown to be beneficial. In addition, it should be a valuable molecule in further studies aiming at precising the structure and function of the IL-11 receptors.

### EXAMPLE 2

### In Vivo Radioprotection

Mice. C₅₇B1/6 male mice, 8-12 weeks old, were purchased from Charles River Laboratories (Chatillon sur Chalaronne, France). Mice were housed at the animal core facility of INSERM U463 at Nantes (France). This facility is approved by the Préfecture of the French Department of Loire-Atlantique and is maintained in accordance with the regulations and standards of French Veterinary Services.

Radiation and Il-11 treatment. Whole body irradiation was delivered with a Teratron 780 (Atomic Energy of Canada limited, Canada) operating ⁶⁰Co sources. The dose rate was 1.5 Gy/min. Human recombinant FPΔIl-11 and H/V-D/A proteins (synthesized by Jean Content (Institut Pasteur, Bruxelles, Belgium) was solubilized in sterile PBS containing 0.2% gelatin, and delivered intravenously by retro-orbital injection of 800 ng, 30 minutes before irradiation and 5, 60 and 120 minutes after irradiation.

Survival studies. Survival as an end point was calculated from the time of treatment until death using the product limit Kaplan-Meier. Differences in product limit Kaplan Meier survival curves were evaluated by the Mantel log-rank test for censored data. Statistical analysis was performed by Student's t test.

Results

One of the major problems encountered by radiotherapists upon irradiation of patients' abdomen is the great radio-sensitivity of the gastrointestinal tract. Local irradiation of the abdomen leads to destruction of the intestinal villae, resulting in dehydration, septic shock and subsequently death of patient. This pathology is known as gastrointestinal syndrome (GI syndrome). It has long been established that death of the stem cells located in the intestinal crypts prevents the regeneration of epithelial cells causing necrosis of the villae (Potten CS, Merritt A, Hickman J, Hall P, Faranda A: Characterization of radiation-induced apoptosis in the small intestine and its biological implications. Int. J. Radiat. Biol. 65:71-8. 1994).

In mice, we observed that a single dose of 15 Gy induced total destruction of the intestinal mucosa and subsequent death of the animal (Paris F, Fuks Z, Kang A, Capodieci P, Juan G, Ehleiter D, Haimovitz-Friedman A, Cordon-Cardo C, Kolesnick R: Endothelial apoptosis as the primary lesion initiating intestinal radiation damage in mice. Science. 293:293-7., 2001).

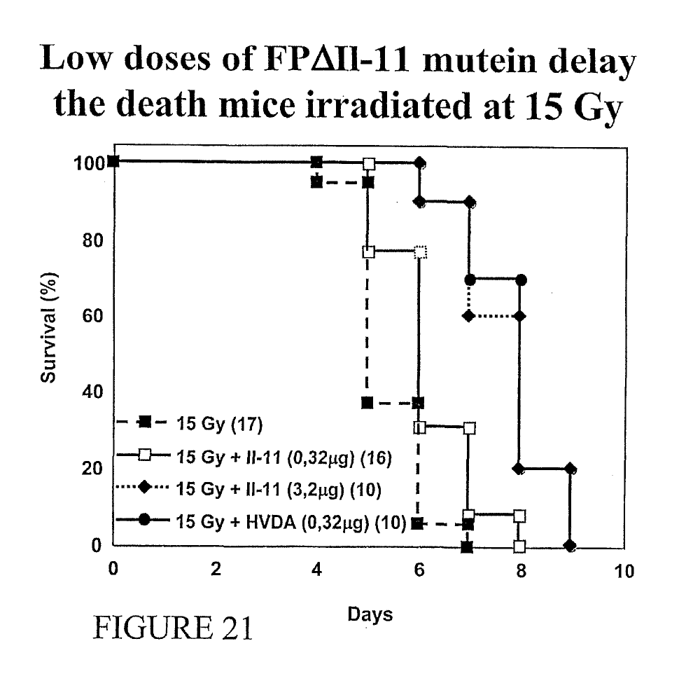

We therefore evaluated the therapeutic potential of FPΔIL-11 (described in the above example 1) in lethally irradiated C57BL6/J mice exposed to γ-rays, and found that FPΔIL-11 delays the death of the animals (median death 8 days for the mice pretreated by the FPΔIL-11 and irradiated versus 5 days for the mice vehicle treated and irradiated at 15 Gy). Results are illustrated by FIG. 21.

In the same experimental conditions, we have evaluated the therapeutic activity of the H/V-D/A mutated proteins. A pretreatment with a 10 time lower dose of H/V-D/A, as compared to the dose used for the FPΔIL-11 (total doses 0,32 μg versus 3,2 μg) delays the mortality to the same iso-effect (median death at 8 days). Results are illustrated by FIG. 22. Pretreatment with FPΔIL-11 at this low dose (0,32 μg.) has only a little impact in survey of the animal irradiated at 15 Gy.

These experiments show that the H/V-D/A mutein of the invention provides with a gain of function, as compared to the wild type IL-11, and improved the protection of the small intestines after exposure to radiation.

### EXAPLE 3

### Comparative Results [Single Point Mutation vs. Double Point Mutation]

Quantification of H/V-D/A hIL-11 Muteins in the blood serum

H/V-D/A IL-11 muteins are injected retroorbitally into the mice 30 minutes prior, 5 minutes, 1 hour and 2 hours after irradiation at 15 Gy.

In a first step, we determine the quantity of H/V-D/A IL-11 that is present in the blood after retroobital injection.

To that end, an ELISA immunoassay is designed to measure H/V-D/A IL-11 in the blood serum collected 30 minutes after injection of 800 ng of the mutein. 96-well plates have been pre-coated with an anti-human IL-11 antibody (polyclonal goat IgG available from R&D Systems under reference AF-218-NA). Standards with recombinant H/V-D/A IL-11 muteins or blood serum are then added to the appropriate microtiter plate wells and incubated.

After washing to remove unbound IL-11 and other components of the sample, anti-Flag antibody against the Flag tag of the H/V-D/A IL-11 muteins (M2, Sigma) and biotin-conjugated polyclonal antibody against the M2 antibody are added and incubated. IL-11, if present, binds to and become immobilized by the antibody pre-coated on the wells and is then “sandwiched” by the biotin conjugate.

In order to quantitatively determine the amount of IL-11 present in the sample, Avidin conjugated to Horseradish Peroxidase (HRP) is added to each microplate well and incubated. The enzyme substrate reaction terminated by the addition of a sulphuric acid solution and the colour change is measured spectrophotometrically at a wavelength of 450 nm.

By this ELISA immunoassay, we detect H/V-DA IL-11 in the serum at the molarity of 2.2 nM, which represent a concentration of 3.3 ng H/V-D/A hIL-11 by ml of blood (the volume of blood in the mice is around 5 ml; and we injected 800 ng of H/V-D/A hIL-11 (or 160 ng/ml of blood) into the mice).

Our data show that 30 minutes after injection, 2% of the muteins are circulating in the blood. The rest has undergone degradation or has bound to their cell receptors.

Effect of D/A Muteins

Single point D/A mutation hIL-11 muteins have been produced in accordance with the teaching of Czupryn et al. 1995 (cited supra), and compared to wild type FPΔIL-11 proteins and to the double point H/V-D/A hIL-11 muteins of the invention:
first in vitro by proliferation induction assay on 7TD1 cells, and
secondly in vivo by studying small intestines radioprotection and death of the animals.

Proliferation assays are made by incubating 7TD1 cells at the concentration of 2×10³ cells in flat bottom 90-well plate in presence of an increasing dose of wild type FPΔIL-11 or its muteins. After 7 days, the number of surviving cells was determined by colorometric assay for hexosamidase.

We found that DA and H/V-D/A hIL-11 have comparable dose response to cell proliferation, whereas wild type recombinant IL-11 is less active (cf. FIG. 35).

Survival of the mice injected retroorbitally with D/A IL-11 muteins after irradiation at 15 Gy are analyzed and compared to survival of the mice injected with wild-type FPΔIL-11 and with the H/N-D/A muteins of the invention (cf. FIG. 36).

Whole body irradiation on 8-12 week-old C₅₇B1/6 male mice is delivered with a Teratron 780 (Atomic Energy of Canada limited, Canada) operating ⁶⁰Co sources. The dose rate is 1.5 Gy/min. Human recombinant FPΔIl-11, D/A and H/V-D/A muteins are solubilized in sterile PBS containing 0.2% gelatin, and delivered intravenously by retro-orbital injection of 800 ng, 30 minutes before irradiation and 5, 60 and 120 minutes after irradiation.

Survival as an end point is calculated from the time of treatment until death using the product limit Kaplan-Meier. Differences in product limit Kaplan Meier survival curves are evaluated by the Mantel log-rank test for censored data. Statistical analysis is performed by Student's t test. In the same experimental conditions, we evaluated the therapeutic activity of the H/V-D/A mutated proteins of the invention as compared to the D/A muteins.

We already demonstrated that a pretreatment with a 10-time lower dose of H/V-D/A, as compared to the dose used for the FPΔIL-11 (total doses 0,32 μg versus 3,2 jig), delays the mortality to the same iso-effect (median death at 8 days); cf. example 2 above. In the same experimental conditions, the injection of 0,32 μg of H/V-D/A muteins delays the mortality of the mice, but not injection of the same dose of D/A muteins. Mice injected with 0,32 μg and irradiated at 15 Gy died quickly with the same iso-effect than mice injected with the same low dose of wild type FPΔIL-11 and irradiated at the same dose.

These experiments show that if the H/V-D/A mutein of the invention provides with a gain of function, as compared to the wild type IL-11 and improved the protection of the small intestines after exposure to radiation, D/A mutein does not provide the same therapeutic gain.

1. A method to produce an IL-11 agonist, which comprises producing a protein having the sequence of an IL-11 mutein that is derivable from a wild-type IL-11 sequence by replacement of at least two non-hydrophobic amino acids within the epitope for IL-11Rα by hydrophobic ones.

2. An IL-11 mutein, the sequence of which comprises a sequence which is derivable from the complete sequence of a wild-type IL-11:
by replacement of the hydrophilic amino acids at positions 182 and 186 (positions computed by reference to the complete wild-type sequence) by X₁ and X₂ respectively, X₁ and X₂ being chosen from the group comprising:
Valine (symbol=V or Val),
Alanine (symbol=A or Ala),
Proline (symbol=P or Pro),
Leucine (symbol=L or Leu),
Isoleucine (symbol=I or Ile),
Phenylalanine (symbol=F or Phe),
Methionine (symbol=M or Met), and
Tryptophan (symbol=W or Trp),
and by deletion of a N-terminal portion that does not exceed the first 34 N-terminal amino acids.

3. The IL-11 mutein of claim 2, wherein said wild-type IL-11 has the sequence of a human IL-11, or of a macaque IL-11, or of a mouse IL-11, or of a rat IL-11.

4. The IL-11 mutein of claim 2, the sequence of which comprises a sequence chosen from the group comprising SEQ ID NO:9, SEQ ID NO:24, SEQ ID NO:39, SEQ ID NO:54, and the conservative variant sequences thereof, wherein said conservative variant sequences are of at least 80%, preferably at least 90% identity with at least one of SEQ ID NO:9, SEQ ID NO:24, SEQ ID NO:39, or SEQ ID NO:54 and provided that the resulting variant protein has retained the ability to induce proliferation of an IL-11 dependent cell line.

5. The IL-11 mutein according to claim 2, wherein X1 and X2 are V or A.

6. The IL-11 mutein according to claim 2, wherein X1=V and X2=A.

7. The IL-11 mutein of claim 6, which comprises a sequence of SEQ ID NO:10, or of SEQ ID NO:25, or of SEQ ID NO:40, or of SEQ ID NO:55.

8. The IL-11 mutein according to claim 2, wherein X1=A and X2=V.

9. The IL-11 mutein of claim 8, which comprises a sequence of SEQ ID NO:11, of SEQ ID NO:26, of SEQ ID NO:41, or of SEQ ID NO:56.

10. The IL-11 mutein according to claim 2, wherein X1=V and X2=V.

11. The IL-11 mutein of claim 10, which comprises a sequence of SEQ ID NO:12, of SEQ ID NO:27, of SEQ ID NO:42, or of SEQ ID NO:57.

12. The IL-11 mutein according to claim 2, wherein X1=A and X2=A.

13. The IL-11 mutein of claim 12, which comprises a sequence of SEQ ID NO:13, of SEQ ID NO:28, of SEQ ID NO:43, or of SEQ ID NO:58.

14. The IL-11 mutein according to claim 2, which comprises a sequence which is derivable from the complete sequence of a wild-type IL-11:
by replacement of the hydrophilic amino acids in positions 182 and 186 (positions computed by reference to the complete wild-type sequence) by X₁ and X₂ respectively, and
by deletion of the first 21 N-terminal amino acids.

15. The IL-11 mutein according to claim 14, which comprises a sequence of SEQ ID NO:14, SEQ ID NO:29, SEQ ID NO:44 or SEQ ID NO:59.

16. The IL-11 mutein according to claim 14, which comprises a sequence of SEQ ID NO:14, SEQ ID NO:29, SEQ ID NO:44 or SEQ ID NO:59, and wherein X₁=V and X₂=A.

17. The IL-11 mutein according to claim 16, which comprises a sequence of SEQ ID NO:15, or of SEQ ID NO:30, or of SEQ ID NO:45, or of SEQ ID NO:60.

18. The IL-11 mutein according to claim 14, which comprises a sequence of SEQ ID NO:14, SEQ ID NO:29, SEQ ID NO:44 or SEQ ID NO:59, and wherein X₁=A and X₂=V.

19. The IL-11 mutein according to claim 16, which comprises a sequence of SEQ ID NO:16, or of SEQ ID NO:31, or of SEQ ID NO:46, or of SEQ ID NO:61.

20. The IL-11 mutein according to claim 14, which comprises a sequence of SEQ ID NO:14, SEQ ID NO:29, SEQ ID NO:44 or SEQ ID NO:59, and wherein X₁=V and X₂=V.

21. The IL-11 mutein according to claim 20, which comprises a sequence of SEQ ID NO:17, or of SEQ ID NO:32, or of SEQ ID NO:47, or of SEQ ID NO:62.

22. The IL-11 mutein according to claim 14, which comprises a sequence of SEQ ID NO:14, SEQ ID NO:29, SEQ ID NO:44 or SEQ ID NO:59, and wherein X₁=A and X₂=A.

23. The IL-11 mutein according to claim 22, which comprises a sequence of SEQ ID NO:18, or of SEQ ID NO:33, or of SEQ ID NO:48, or of SEQ ID NO:63.

24. The IL-11 mutein according to claim 2, which comprises a sequence which is derivable from the complete sequence of a wild-type IL-11, by replacement of the hydrophilic amino acids in positions 182 and 186 (positions computed by reference to the complete wild-type sequence) by X₁ and X₂ respectively.

25. The IL-11 mutein according to claim 24, which comprises a sequence of SEQ ID NO:19, or of SEQ ID NO:34, or of SEQ ID NO:49, or of SEQ ID NO:64.

26. The IL-11 mutein according to claim 24, wherein X₁=V and X₂=A.

27. The IL-11 mutein according to claim 24, wherein X₁=V and X₂=A, and which comprises a sequence of
SEQ ID NO:20, or of SEQ ID NO:35, or of SEQ ID NO:50, or of SEQ ID NO:65.

28. The IL-11 mutein according to claim 24, wherein X₁=A and X₂=V.

29. The IL-11 mutein according to claim 24, wherein X₁=A and X₂=V, and which comprises a sequence of SEQ ID NO:21, or of SEQ ID NO:36, or of SEQ ID NO:51, or of SEQ ID NO:66.

30. The IL-11 mutein according to claim 24, wherein X₁=V and X₂=V.

31. The IL-11 mutein according to claim 24, wherein X₁=V and X₂=V, and which comprises a sequence of SEQ ID NO:22, or of SEQ ID NO:37, or of SEQ ID NO:52, or of SEQ ID NO:67.

32. The IL-11 mutein according to claim 24, wherein X₁=A and X₂=A.

33. The IL-11 mutein according to claim 24, wherein X₁=A and X₂=A and which comprises a sequence of SEQ ID NO:23, or of SEQ ID NO:38, or of SEQ ID NO:53, or of SEQ ID NO:68.

34. A nucleic acid, wherein its sequence codes for a mutein according to claim 2.

35. The nucleic acid according to claim 34, which comprises the sequence of SEQ ID NO:72, wherein each of n₁n₂n₃ and n₄n₅n₆ codes for:
Valine (symbol=V or Val), or
Alanine (symbol=A or Ala), or
Proline (symbol=P or Pro), or
Leucine (symbol=L or Leu), or
Isoleucine (symbol=I or Ile), or
Phenylalanine (symbol=F or Phe), or
Methionine (symbol=M or Met), or
Tryptophan (symbol=W or Trp).

36. The nucleic acid according to claim 34, which comprises the sequence of SEQ ID NO:72, wherein n₁n₂n₃ and n₄n₅n₆ are both selected from the group comprising the following codons:
GCT, GCC, GCA, GCG,
GTT, GTC, GTA, GTG,
TTA, TTG, CTT, CTC, CTA, CTG,
ATT, ATC, ATA,
TTT, TTC,
ATG,
CCT, CCC, CCA, CCG,
TGG.

37. The nucleic acid according to claim 34, which comprises the sequence of SEQ ID NO:71 or of SEQ ID NO:70.

38. The nucleic acid according to claim 34, which comprises the sequence of SEQ ID NO:76 or of SEQ ID NO:74.

39. The nucleic acid according to claim 34, which has the RNA sequence of SEQ ID NO:75, wherein the codons n₁n₂n₃ and n₄n₅n₆ are both selected from the group comprising the following codons:
GCU, GCC, GCA, GCG
GUU, GUC, GUA, GUG,
UUA, UUG, CUU, CUC, CUA, CUG,
AUU, AUC, AUA,
UUU, UUC,
AUG,
CCU, CCC, CCA, CCG,
UGG.

40. A transfection vector, which comprises a nucleic acid according to claim 34.

41. The transfection vector according to claim 40, which further comprises a nucleotide sequence coding for a Flag tag.

42. The transfection vector according to claim 40, which comprises the sequence of SEQ ID NO:79.

43. A cell, which comprises a nucleic acid according to claim 34, or which has been transfected by a tranfection vector or which express a mutein.

44. A drug which comprises:
a therapeutically effective amount of an IL-11 mutein according to claim 2, or of a nucleic acid or of a transfection vector or of a cell,
and, optionally, a pharmaceutically-acceptable vehicle.

45. The drug according to claim 44, which is intended for the prevention or treatment of an inflammatory disease or condition.

46. The drug according to claim 44, which is intended for the prevention or treatment of a septic shock.

47. The drug according to claim 44, which is intended for the prevention or treatment of diabetes.

48. The drug according to claim 44, which is intended for inhibiting microvascular endothelium apoptosis.

49. The drug according to claim 44, which is an anti-thrombocytopenia drug.
